(19) United States
(12) Patent Application Publication
(10) Pub. No.: US 2007/0105836 A1
(43) Pub. Date: May 10, 2007

(54) Prodrugs of muscarinic agonists and methods of treatment of neuropsychiatric disorders

(76) Inventors: Lars Pettersson, Skogsvagen (SE); Gilles Gaubert, Malmo (SE); Jorgen Ohlsson, Sandby (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/590,559

(22) Filed: Oct. 31, 2006

Related U.S. Application Data
(60) Provisional application No. 60/731,948, filed on Oct. 31, 2005.

(51) Int. Cl. A61K 31/553; A61K 31/551; A61K 31/55; C07D 413/04; C07D 403/04

(52) U.S. Cl. 514/211.13; 514/217; 514/220; 540/551; 540/557; 540/587

(57) ABSTRACT

Compounds are described that are prodrugs to active compounds that modulate a muscarinic receptor. In some cases, the compounds are prodrugs to N-desmethylclozapine. The compounds may be used to treat neuropsychiatric disorders.

### RELATED APPLICATION

This application claims the benefit of U.S. Provisional Application No. 60/731,948, filed Oct. 31, 2005, which is incorporated herein by reference in its entirety.

### BACKGROUND OF THE INVENTION

1. Field of the Invention

Certain aspects of the present disclosure relate to methods for treatment of neuropsychiatric disorders, pain, and other disorders by compounds that modulate the activity of muscarinic receptors (e.g., the subtype M1), thereby modulating neuronal activities associated with the development of neuropsychiatric disorders. Aspects of the invention also relate to compounds that act as prodrugs, releasing active compounds that selectively interact with this receptor subtype.

2. Description of the Related Art

Muscarinic cholinergic receptors mediate the actions of the neurotransmitter acetylcholine in the central and peripheral nervous systems, gastrointestinal system, heart, endocrine glands, lungs, and other tissues. Muscarinic receptors play a central role in the central nervous system for higher cognitive functions, as well as in the peripheral parasympathetic nervous system. Five distinct muscarinic receptor subtypes have been identified, m1-m5. The m1 subtype is the predominant subtype found in the cerebral cortex and is believed to be involved in the control of cognitive functions; m2 is the predominant subtype found in heart and is believed to be involved in the control of heart rate; m3 is believed to be involved in gastrointestinal and urinary tract stimulation as well as sweating and salivation; m4 is present in brain and may be involved in locomotion; and m5, present in brain, may be involved in certain functions of the central nervous system associated with the dopaminergic system.

Conditions associated with cognitive impairment, such as Alzheimer's disease, are accompanied by loss of acetylcholine in the brain. This is believed to be the result of degeneration of cholinergic neurons in the basal forebrain, which innervate areas of the association cortex, and hippocampus, which is involved in higher processes.

Efforts to increase acetylcholine levels have focused on increasing levels of choline, the precursor for acetylcholine synthesis, and on blocking acetylcholine esterase (AChE), the enzyme that metabolizes acetylcholine. Administration of choline or phosphatidylcholine has not been very successful. AChE inhibitors have shown some therapeutic efficacy, but may cause cholinergic side effects due to peripheral acetylcholine stimulation, including abdominal cramps, nausea, vomiting, diarrhea, anorexia, weight loss, myopathy and depression. Gastrointestinal side effects have been observed in about a third of the patients treated. In addition, some AChE inhibitors, such as tacrine, have also been found to cause significant hepatotoxicity, with elevated liver transaminases observed in about 30% of patients. The adverse effects of AChE inhibitors have limited their clinical utility.

Known muscarinic agonists such as arecoline, pilocarpine and oxotremorine have also been found to be agonists at all 5 muscarinic receptor subtypes and are not very effective in treating cognitive impairment, most likely because of dose-limiting side effects.

There is a need for compounds that increase acetylcholine signaling or effect in the brain. Specifically there is a need for muscarinic agonists that are active at various muscarinic receptor subtypes in the central and peripheral nervous system. Furthermore, there is a need for more highly selective muscarinic agonists, such as m1- or m4-selective agents, both as pharmacological tools and as therapeutic agents.

### SUMMARY OF THE INVENTION

One embodiment disclosed herein includes a compound of formula I:

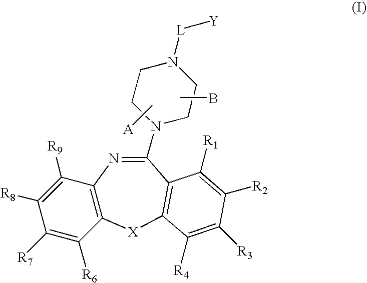

or a pharmaceutically acceptable salt thereof, wherein:

X is selected from the group consisting of NR¹⁰, O, CR¹⁰R¹¹, C═CR¹⁰R¹¹, and C═O;

A and B are independently selected from the group consisting of hydrogen and mono-substituted, poly-substituted or unsubstituted, cyclic or acyclic, straight or branched -chain variants of the following residues: C₁-C₆ alkyl, C₂-C₆ alkenyl, C₂-C₆ alkynyl, and C₂-C₆ alkoxy;

A and B may optionally be bound together to form a cycloalkyl, heterocyclyl, heteroaryl, or aryl ring fused to the piperazine ring;

L is a cleavable linker moiety capable of being metabolically cleaved from the piperazine nitrogen;

Y is selected from the group consisting of hydrogen; halogen; cyano; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂, —NR¹⁰COR¹¹; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, acyl, arylalkyl, heteroarylalkyl, alkyloxycarbonyloxy, arylalkoxy, C₁-C₂₄ alkoxyalkyl, C₁-C₂₄ alkylthio, C₃-C₂₄ heterocycloalkyl-alkyl, and C₃-C₂₄ heterocycloalkenyl-alkyl; and mono-substituted, poly-substituted or unsubstituted variants of the following residues: acyloxy, aryloxycarbonyloxy, C₃-C₂₄ cycloalkyl, C₃-C₂₄ cycloalkenyl, C₂-C₂₄ cycloalkoxy, aryl, heteroaryl, C₂-C₂₄ heterocycloalkyl, C₂-C₂₄ heterocycloalkenyl, carbonyl, amino, aminocarbonyl, aminocarbonyloxy, nitro, azido, phenyl, hydroxyl, arylthio, carboxy, thio, acyl mono-radicals derived from naturally occurring amino acids or from lipoic acid, —CH₂CH₂SC(O)OR¹⁰, and —CH₂CH₂SC(O)R¹⁰, with the proviso that -L-Y is not unsubstituted alkyl;

R¹, R², R³, R⁴, R⁶, R⁷, R⁸, and R⁹ are each independently selected from the group consisting of hydrogen; halogen; —OH; —SH; —CN; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂; —NR¹⁰C(O)R¹¹; and mono-substituted, poly-substituted or unsubstituted, straight or branched chain variants of the following residues: C₁₋₆ alkyl, C₂₋₆ alkenyl, C₂₋₆ alkynyl, C₁₋₆ alkyloxy, C₁₋₆ heteroalkyl, C₁₋₆-alkoxyalkyl, C₁₋₆ alkylthio, C₁₋₆ perhaloalkyl, and C₁₋₆ perhaloalkoxy;

independently R¹ and R², R² and R³, R³ and R⁴, R⁶ and R⁷, R⁷ and R⁸, or R⁸ and R⁹ may optionally be taken together, along with the ring carbons to which they are attached, to form a five-membered or six-membered cycloalkyl, heterocyclyl, or heteroaryl ring, or a six-membered aryl ring;

independently R¹⁰ in X may optionally be bound to R⁴ or R⁶ to form a five-membered optionally substituted heterocyclyl or heteroaryl ring system; and

R¹⁰, R¹¹, and R¹² are each independently selected from a group consisting of hydrogen and mono-substituted, poly-substituted or unsubstituted, straight or branched chain variants of the following residues: C₁₋₆ alkyl, C₃₋₆ cycloalkyl, C₂₋₆ alkenyl, C₂₋₆ alkynyl, arylalkyl, alkylcarbonyl, C₂₋₆ alkoxycarbonyl, C₆₋₁₀ aryl, and C₅₋₁₀ heteroaryl.

In one embodiment, X is not selected from one or more of NR¹⁰, O, CR¹⁰R¹¹, C═CR¹⁰R¹¹, and C═O.

In one embodiment, A and/or B are not selected from one or more of hydrogen and mono-substituted, poly-substituted or unsubstituted, cyclic or acyclic, straight or branched chain variants of the following residues: C₁-C₆ alkyl, C₂-C₆ alkenyl, C₂-C₆ alkynyl, and C₂-C₆ alkoxy.

In one embodiment, Y is not selected from one or more of hydrogen; halogen; cyano; —C(O)R¹⁰; —C(O)OR¹⁰; —C(O)NR¹⁰R¹¹; —NR¹²C(O)NR¹⁰R¹¹; —SO₂NR¹⁰R¹¹, —SO₂R¹⁰; —OSO₂R¹⁰; —NO₂, —NR¹⁰COR¹¹; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, acyl, arylalkyl, heteroarylalkyl, alkyloxycarbonyloxy, arylalkoxy, C₁-C₂₄ alkoxyalkyl, C₁-C₂₄ alkylthio, C₃-C₂₄ heterocycloalkyl-alkyl, and C₃-C₂₄ heterocycloalkenyl-alkyl; and mono-substituted, poly-substituted or unsubstituted variants of the following residues: acyloxy, aryloxycarbonyloxy, C₃-C₂₄ cycloalkyl, C₃-C₂₄ cycloalkenyl, C₂-C₂₄ cycloalkoxy, aryl, heteroaryl, C₂-C₂₄ heterocycloalkyl, C₂-C₂₄ heterocycloalkenyl, carbonyl, amino, aminocarbonyl, aminocarbonyloxy, nitro, azido, phenyl, hydroxyl, arylthio, carboxy, thio, acyl mono-radicals derived from naturally occurring amino acids or from lipoic acid, —CH₂CH₂SC(O)OR¹⁰, and —CH₂CH₂SC(O)R¹⁰.

In one embodiment, R¹, R², R³, R⁴, R⁶, R⁷, R⁸, and/or R⁹ are not selected from one or more of hydrogen; halogen; —OH; —SH; —CN; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂; —NR¹⁰C(O)R¹¹; and mono-substituted, poly-substituted or unsubstituted, straight or branched chain variants of the following residues: C₁₋₆ alkyl, C₂₋₆ alkenyl, C₂₋₆ alkynyl, C₁₋₆ alkyloxy, C₁₋₆ heteroalkyl, C₁₋₆-alkoxyalkyl, C₁₋₆alkylthio, C₁₋₆perhaloalkyl, and C₁₋₆perhaloalkoxy.

In one embodiment, R¹ and R², or R² and R³, or R³ and R⁴, or R⁶ and R⁷, or R⁷ and R⁸, or R⁸ and R⁹ independently taken together, along with the ring carbons to which they are attached, do not form a five-membered or six-membered cycloalkyl, heterocyclyl, or heteroaryl ring, or a six-membered aryl ring.

In one embodiment, R¹⁰ in X is not bound to R⁴ or R⁶ to form a five-membered optionally substituted heterocyclyl or heteroaryl ring system

In one embodiment, R¹⁰, R¹¹, and R¹² are not selected from one more of hydrogen and mono-substituted, poly-substituted or unsubstituted, straight or branched chain variants of the following residues: C₁₋₆ alkyl, C₃₋₆ cycloalkyl, C₂₋₆ alkenyl, C₂₋₆ alkynyl, arylalkyl, C₂₋₆ alkylcarbonyl, C₂₋₆ alkoxycarbonyl, C₆₋₁₀ aryl, and C₅₋₁₀ heteroaryl.

In one embodiment, L together with the piperzine nitrogen to which it is attached do not form one or more of a tertiary amine, a carbamate, a urea, an amide, an enamine, a sulfamide, a sulfonamide, an aminal, a hydrazine, or a hydroxylamine. In one embodiment, L is not selected from one or more of —C(O)—, —C(O)O—, —CH₂)₂O—, —C(O)NR¹⁰—, —NR¹⁰C(O)NR¹¹—, —NR¹⁰C(O)—, —NR¹⁰—, —CR¹⁰R¹¹OC(O)—, —CR¹⁰R¹¹C(O)O—,

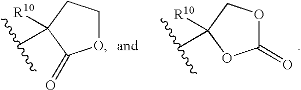

In some embodiments, L is selected such that the piperizine nitrogen together with L form a tertiary amine, a carbamate, a urea, an amide, an enamine, a sulfamide, a sulfonamide, an aminal, a hydrazine, or a hydroxylamine. In some embodiments, L is selected from the group consisting of carboxy, carbonyl, and alkoxy. In some embodiments, L is selected from the group consisting of —C(O)O—, —C(O)—, and —(CH₂)₂O—. In some embodiments, L is selected from a group consisting of —C(O)—, —C(O)O, —C(O)NR¹⁰—, —NR¹⁰C(O)NR¹¹—, —NR¹⁰C(O)—, —NR¹⁰—, —CR¹⁰R¹¹OC(O)—, —CR¹⁰R¹¹C(O)O,

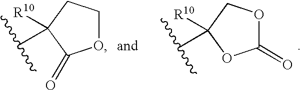

In some embodiments, Y is selected from a group consisting of hydrogen; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, phenyl-C₁₋₂-alkyl, C₃-C₂₄ heterocycloalkyl-alkyl, and C₃-C₂₄ heterocycloalkenyl-alkyl; and mono-substituted, poly-substituted or unsubstituted variants of the following residues: C₃-C₂₄ cycloalkyl, C₃-C₂₄ cycloalkenyl, C₂-C₂₄ cycloalkoxy, C₂-C₂₄ heterocycloalkyl, C₂-C₂₄ heterocycloalkenyl, acyl mono-radicals derived from naturally occurring amino acids or from lipoic acid, —CH₂CH₂SC(O)OR¹⁰, and —CH₂CH₂SC(O)R¹⁰.

In some embodiments, R² is H or F. In some embodiments, R⁸ is Cl, Br, or I. In some embodiments, R⁴ is Cl or Me. In some embodiments, X is NH. In some embodiments, X is O.

In some embodiments, the compound is selected from the group consisting of: 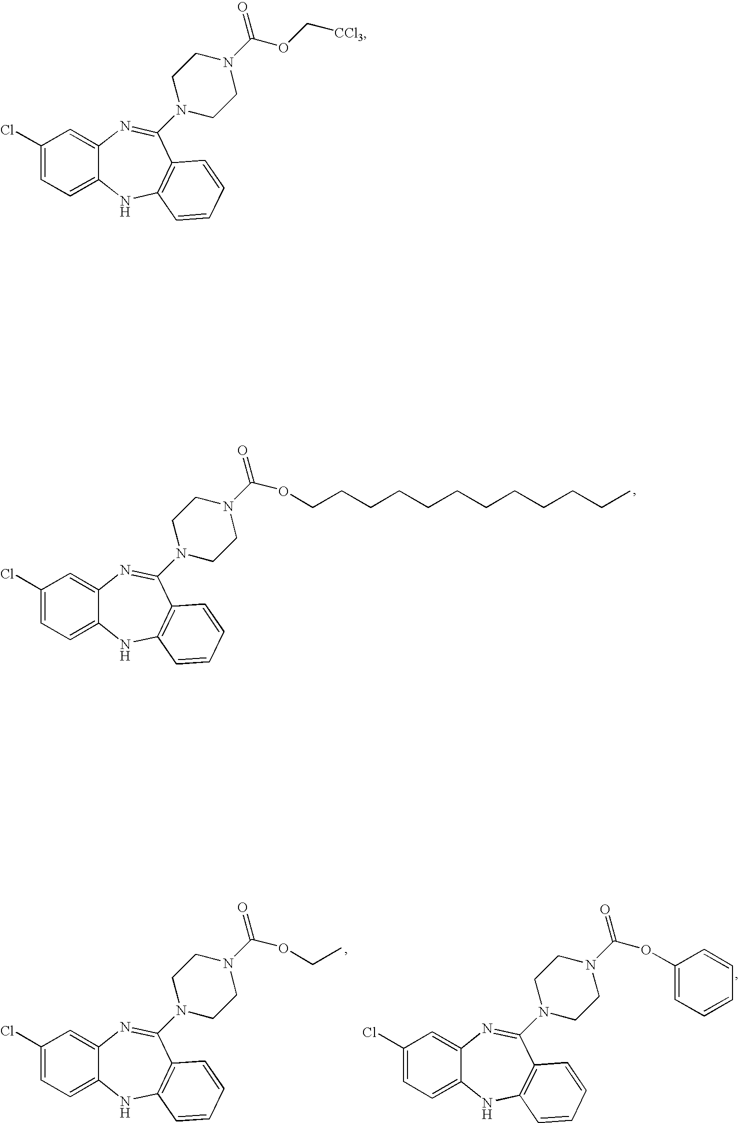 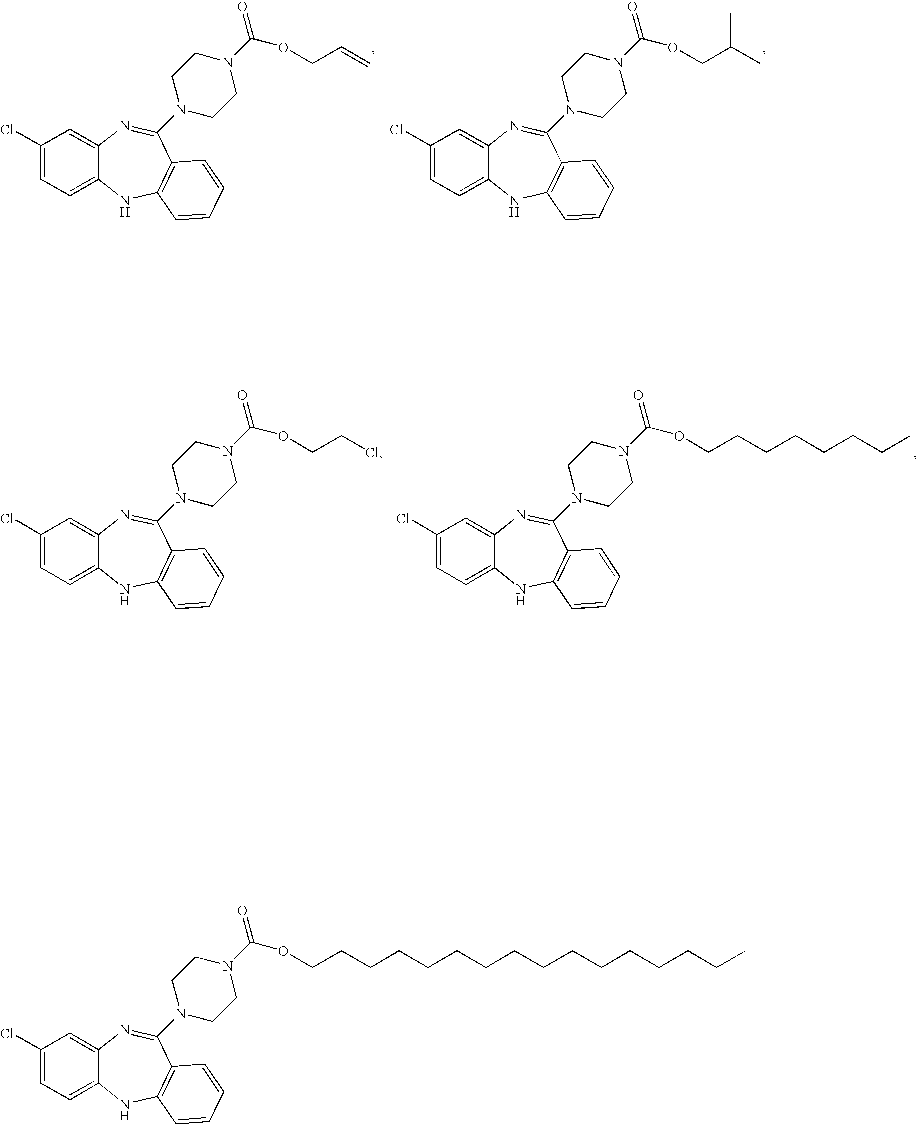 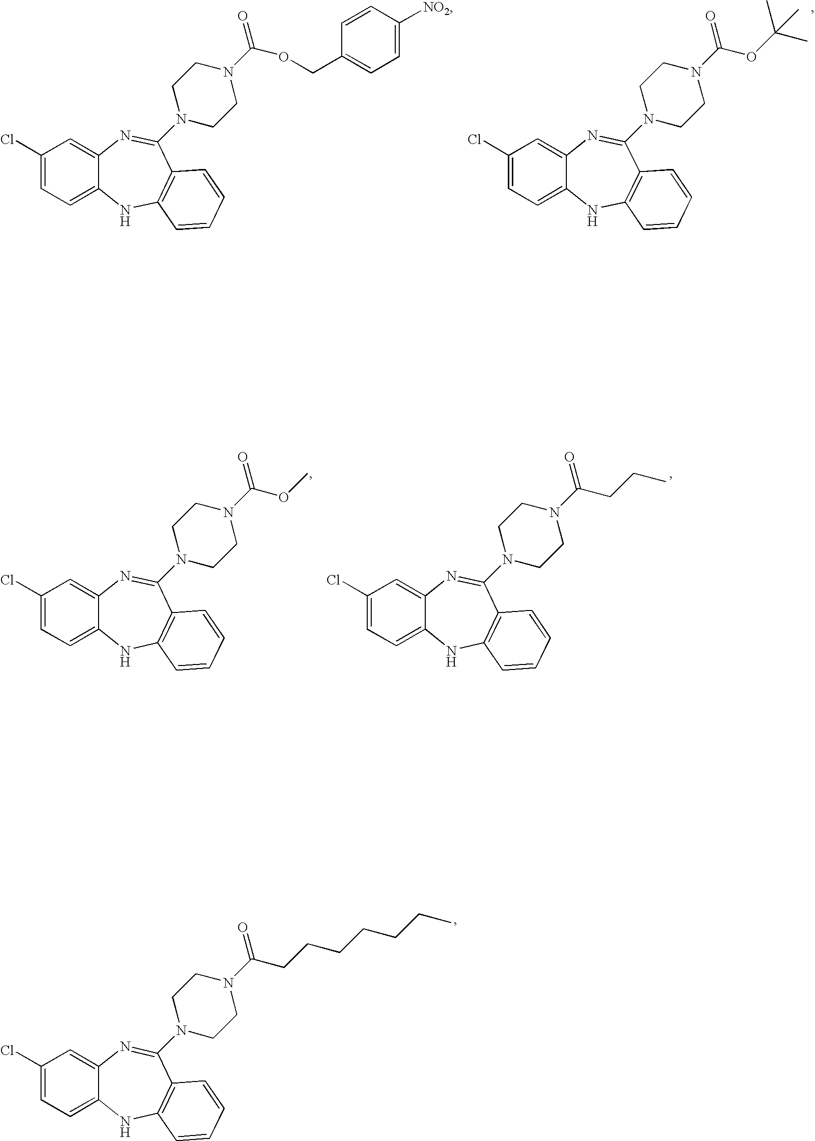 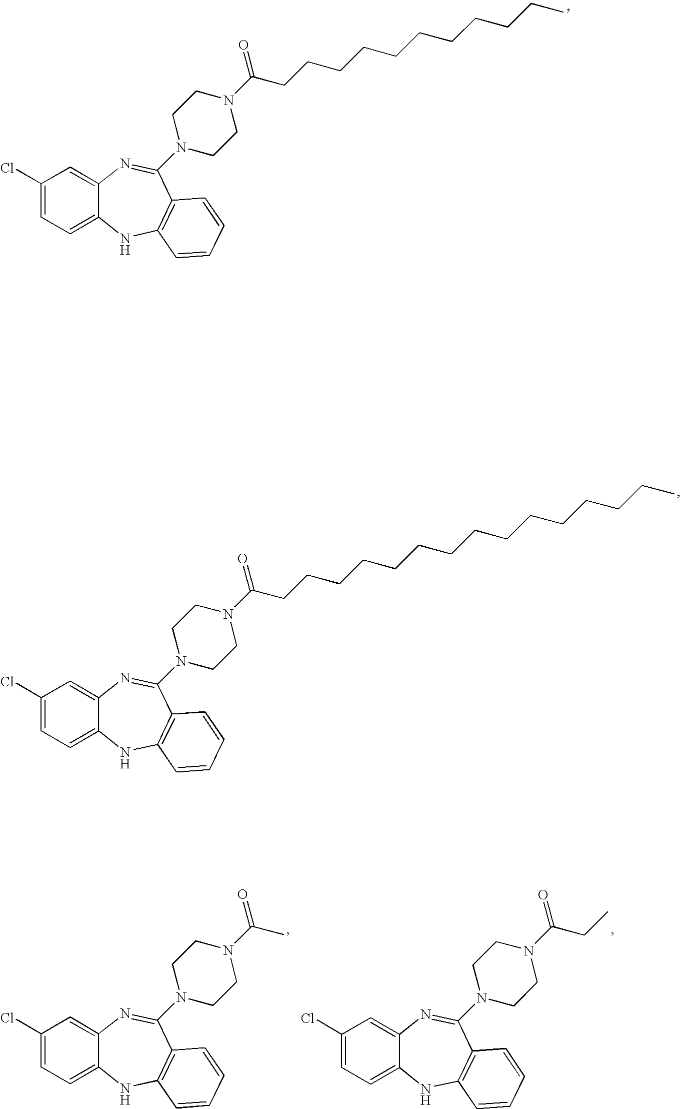 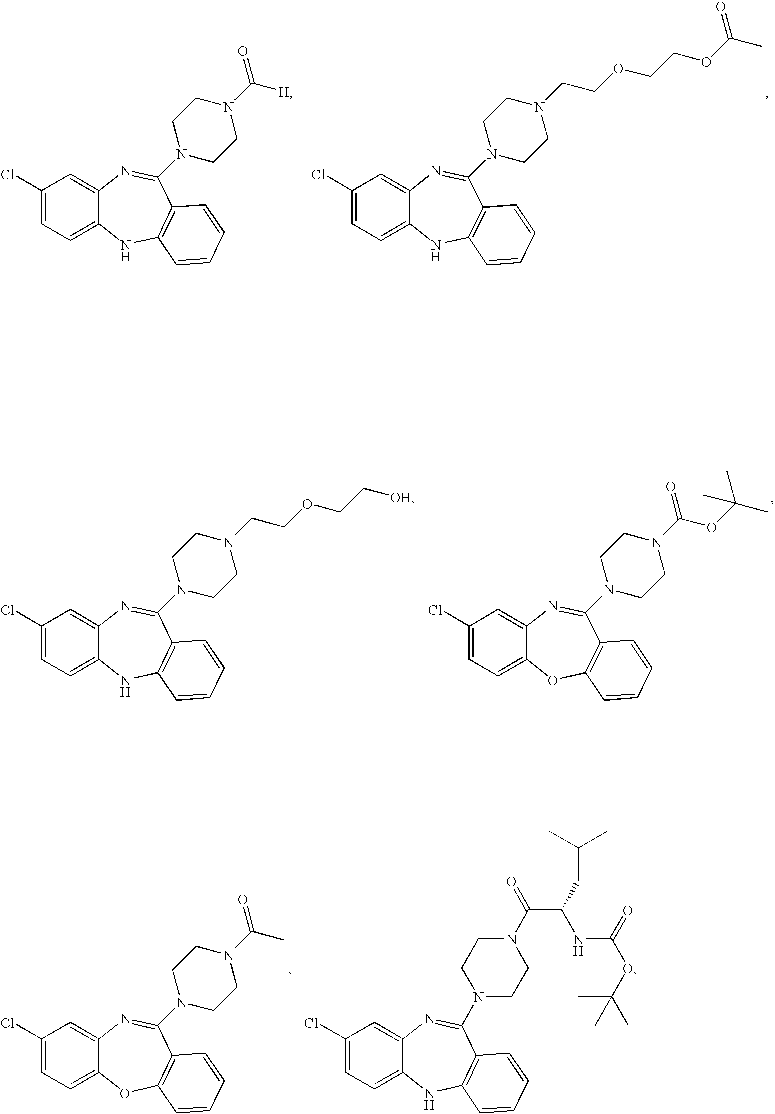 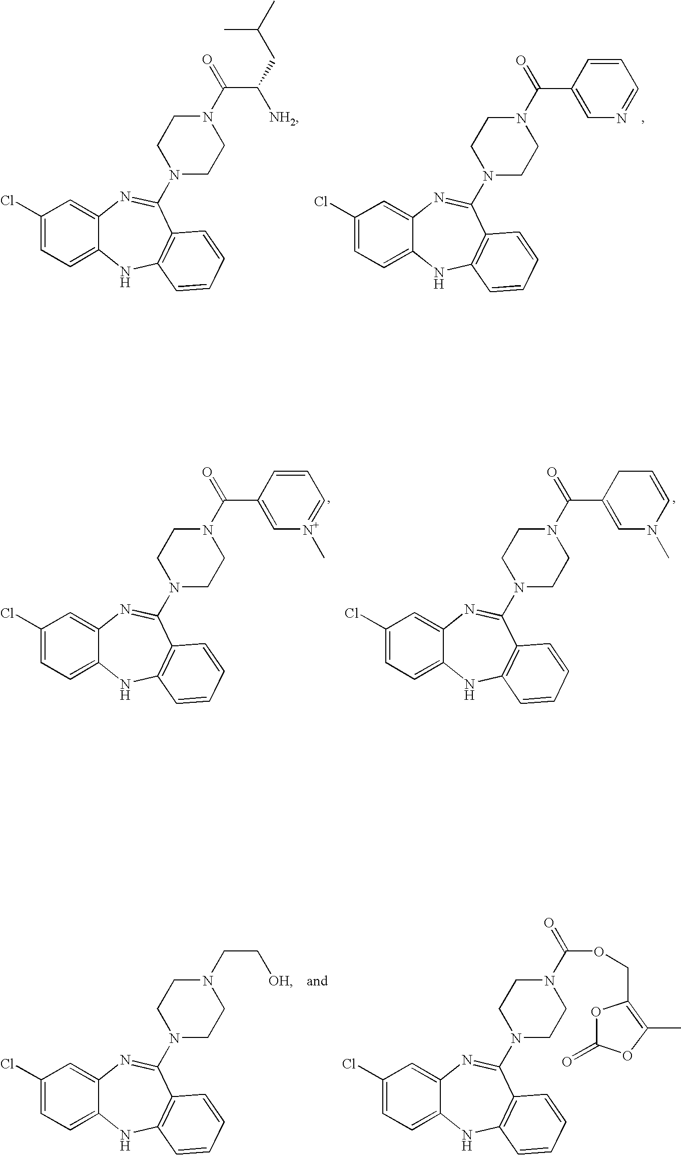
or pharmaceutically acceptable salts thereof

Another embodiment disclosed herein includes a compound of formula (II):

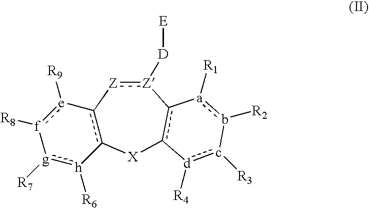

or a pharmaceutically acceptable salt thereof, wherein:

D is absent or is selected from the group consisting of —NH(CH₂)ₙ— and —(CH₂)ₙ—;

B is selected from the group consisting of:

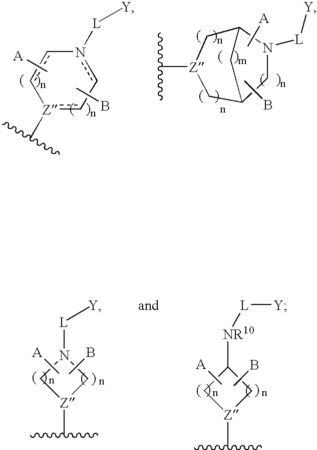

Z is nitrogen, CH, or CH₂;

Z′ is C or CH, wherein when Z′ is C, there is a double bond between Z and Z′ and wherein when Z′ is CH, there is a single bond between Z and Z′;

Z″ is N or CH;

each n is separately selected from the group consisting of 0, 1, 2, 3, and 4;

m is selected from the group consisting of 1, 2, and 3;

X is selected from the group consisting of NR¹⁰, O, CR¹⁰R¹¹, C═CR¹⁰R¹¹, and C═O;

A and B are independently selected from the group consisting of hydrogen and mono-substituted, poly-substituted or unsubstituted, cyclic or acyclic, straight or branched chain variants of the following residues: C₁-C₆ alkyl, C₂-C₆ alkenyl, C₂-C₆ alkynyl, and C₂-C₆ alkoxy;

A and B may optionally be bound together to form a fused cycloalkyl, heterocyclyl, heteroaryl, or aryl ring;

L is a cleavable linker moiety capable of being metabolically cleaved from the nitrogen to which it is attached;

Y is selected from the group consisting of hydrogen; halogen; cyano; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂, —NR¹⁰COR¹¹; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, acyl, arylalkyl, heteroarylalkyl, alkyloxycarbonyloxy, arylalkoxy, C₁-C₂₄ alkoxyalkyl, C₁-C₂₄ alkylthio, C₃-C₂₄ heterocycloalkyl-alkyl, and C₃-C₂₄ heterocycloalkenyl-alkyl; and mono-substituted, poly-substituted or unsubstituted variants of the following residues: acyloxy, aryloxycarbonyloxy, C₃-C₂₄ cycloalkyl, C₃-C₂₄ cycloalkenyl, C₂-C₂₄ cycloalkoxy, aryl, heteroaryl, C₂-C₂₄ heterocycloalkyl, C₂-C₂₄ heterocycloalkenyl, carbonyl, amino, aminocarbonyl, aminocarbonyloxy, nitro, azido, phenyl, hydroxyl, arylthio, carboxy, thio, acyl mono-radicals derived from naturally occurring amino acids or from lipoic acid, —CH₂CH₂SC(O)OR¹⁰, and —CH₂CH₂SC(O)R¹⁰, with the proviso that -L-Y is not unsubstituted alkyl;

a, b, c, and d are each separately selected from the group consisting of carbon, nitrogen, oxygen, and sulfur, or each is separately absent,

provided that at least three of a, b, c, or d are present,

provided that at least one of a, b, c, or d is carbon, and

provided that no two adjacent a, b, c, or d are both oxygen or both sulfur;

e, f, g, and h are each separately selected from the group consisting of carbon, nitrogen, oxygen, and sulfur, or each is separately absent,

provided that at least three of e, f, g, or h are present,

provided that at least one of e, f, g, or h is carbon, and

provided that no two adjacent e, f, g, or h are both oxygen or both sulfur;

R¹, R², R³, R⁴, R⁶, R⁷, R⁸, and R⁹ are each independently selected from the group consisting of hydrogen; halogen; —OH; —SH; —CN; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂; —NR¹⁰C(O)R¹¹; and mono-substituted, poly-substituted or unsubstituted, straight or branched chain variants of the following residues: C₁₋₆ alkyl, C₂₋₆ alkenyl, C₂₋₆ alkynyl, C₁₋₆ alkyloxy, C₁₋₆ heteroalkyl, C₁₋₆-alkoxyalkyl, C₁₋₆ alkylthio, C₁₋₆ perhaloalkyl, and C₁₋₆ perhaloalkoxy, independently R¹ and R², R² and R³, R³ and R⁴, R⁶ and R⁷, R⁷ and R⁸, or R⁸ and R⁹ may optionally be taken together, along with the ring carbons to which they are attached, to form a five-membered or six-membered cycloalkyl, heterocyclyl, or heteroaryl ring, or a six-membered aryl ring,

independently R¹⁰ in X may optionally be bound to R⁴ or R⁶ to form a five-membered optionally substituted heterocyclyl or heteroaryl ring system;

R¹⁰, R¹¹, and R¹² are each independently selected from a group consisting of hydrogen and mono-substituted, poly-substituted or unsubstituted, straight or branched chain variants of the following residues: C₁₋₆ alkyl, C₃₋₆ cycloalkyl, C₂₋₆ alkenyl, C₂₋₆ alkynyl, arylalkyl, alkylcarbonyl, C₂₋₆ alkoxycarbonyl, C₆₋₁₀ aryl, and C₅₋₁₀ heteroaryl; and

any bond represented by a dashed and solid line represents a bond selected from the group consisting of a carbon-carbon single bond and a carbon-carbon double bond.

Another embodiment disclosed herein includes a method of modulating the activity of a muscarinic receptor, comprising administering to a subject a compound of formula I or II. In some embodiments, administration of the compound causes activation of the muscarinic receptor. In some embodiments, administration of the compound causes inhibition of the muscarinic receptor. In some embodiments, administration of the compound causes formation of a second compound in vivo that is an agonist of the muscarinic receptor. In some embodiments, administration of the compound causes formation of a second compound in vivo that is an antagonist of the muscarinic receptor.

Another embodiment disclosed herein includes a method of treating or preventing a neuropsychiatric disorder, comprising administering to a subject a compound of formula I or II. In some embodiments, the neuropsychiatric disorder is selected from the group consisting of one or more of psychosis, cognitive impairment associated with psychosis, hallucination, delusion, disordered thought, behavioral disturbance, aggression, neuropathic pain, anhedonia, a psychiatric disturbance secondary to dementia or cognitive impairment, a behavioral disturbance secondary to dementia or cognitive impairment, schizophrenia, an idiopathic psychosis, anxiety, a sleep disorder, an appetite disorder, an affective disorder, Tourette's Syndrome, drug-induced psychosis, psychosis secondary to a neurodegenerative disorder, and cognitive impairment secondary to a neurodegenerative disorder. In some embodiments, the affective disorder is selected from one or more of major depression, bipolar disorder, mania, flattening of affect, suicidality, and depression with psychotic features. In some embodiments, the neurodegenerative disorder is selected from one or more of Alzheimer's and Huntington's disease.

Another embodiment disclosed herein includes a method of treating or preventing glaucoma, comprising administering to a subject a compound of formula I or II.

Another embodiment disclosed herein includes a pharmaceutical composition, comprising a compound of formula I or II. In some embodiments, the composition comprises one or more of a physiologically acceptable carrier, diluent, or excipient.

### DETAILED DESCRIPTION OF THE PREFERRED EMBODIMENT

As described in PCT Publication Nos. WO2004/073639 and WO2004/064753, which are incorporated herein by reference in their entirety, N-desmethylclozapine is an ectopic activator of the muscarinic receptor subtype M1 and, as such, is useful for treatment of neuropsychiatric diseases. It has also been shown that N-desmethylclozapine is the main metabolite, and probably the predominant active principle, of clozapine when used as a atypical anti-psychotic agent. Thus, clozapine can be seen as a prodrug of N-desmethylclozapine. However, clozapine itself displays other pharmacological activities that may cause unwanted side-effects.

Prodrugs are commonly used to enhance the pharmacokinetic behavior of drugs. Prodrugs are a protected form of the parent drug and are converted by metabolic processes to the parent drug. Parameters that can be affected by this approach are e.g. solubility, dissolution, absorption, bioavailability, metabolism, tissue penetration and distribution, etc., which may enhance the pharmacodynamic profile of the drug. e.g. in terms of adverse effects.

Accordingly, in one embodiment, a compound of Formula I is provided:

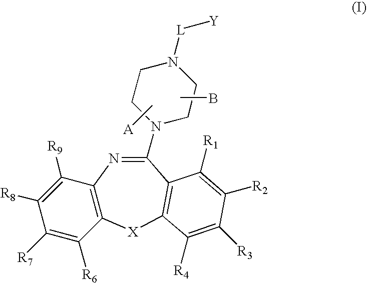

or a pharmaceutically acceptable salt thereof, wherein:

X is selected from the group consisting of NR¹⁰, O, CR¹⁰R¹¹, C═CR¹⁰R¹¹, and C═O;

A and B are independently selected from the group consisting of hydrogen and mono-substituted, poly-substituted or unsubstituted, cyclic or acyclic, straight or branched chain variants of the following residues: C₁-C₆ alkyl, C₂-C₆ alkenyl, C₂-C₆ alkynyl, and C₂-C₆ alkoxy;

A and B may optionally be bound together to form a cycloalkyl, heterocyclyl, heteroaryl, or aryl ring fused to the piperazine ring;

L is a cleavable linker moiety capable of being metabolically cleaved from the piperazine nitrogen;

Y is selected from the group consisting of hydrogen; halogen; cyano; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂, —NR¹⁰COR¹¹; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, acyl, arylalkyl, heteroarylalkyl, alkyloxycarbonyloxy, arylalkoxy, C₁-C₂₄ alkoxyalkyl, C₁-C₂₄ alkylthio, C₃-C₂₄ heterocycloalkyl-alkyl, and C₃-C₂₄ heterocycloalkenyl-alkyl; and mono-substituted, poly-substituted or unsubstituted variants of the following residues: acyloxy, aryloxycarbonyloxy, C₃-C₂₄ cycloalkyl, C₃-C₂₄ cycloalkenyl, C₂-C₂₄ cycloalkoxy, aryl, heteroaryl, C₂-C₂₄ heterocycloalkyl, C₂-C₂₄ heterocycloalkenyl, carbonyl, amino, aminocarbonyl, arninocarbonyloxy, nitro, azido, phenyl, hydroxyl, arylthio, carboxy, thio, acyl mono-radicals derived from naturally occurring amino acids or from lipoic acid, —CH₂CH₂SC(O)OR¹⁰, and —CH₂CH₂SC(O)R¹⁰, with the proviso that -L-Y is not unsubstituted alkyl;

R¹, R², R³, R⁴, R⁶, R⁷, R⁸, and R⁹ are each independently selected from the group consisting of hydrogen; halogen; —OH; —SH; —CN; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂; —NR¹⁰C(O)R¹¹; and mono-substituted, poly-substituted or unsubstituted, straight or branched chain variants of the following residues: C₁₋₆ alkyl, C₂₋₆ alkenyl, C₂₋₆ alkynyl, C₁₋₆ alkyloxy, C₁₋₆ heteroalkyl, C₁₋₆-alkoxyalkyl, C₁₋₆ alkylthio, C₁₋₆ perhaloalkyl, and C₁₋₆ perhaloalkoxy;

independently R¹ and R², R² and R³, R³ and R⁴, R⁶ and R⁷, R⁷ and R⁸, or R⁸ and R⁹ may optionally be taken together, along with the ring carbons to which they are attached, to form a five-membered or six-membered cycloalkyl, heterocyclyl, or heteroaryl ring, or a six-membered aryl ring;

independently R¹⁰ in X may optionally be bound to R⁴ or R⁶ to form a five-membered optionally substituted heterocyclyl or heteroaryl ring system; and

R¹⁰, R¹¹, and R¹² are each independently selected from a group consisting of hydrogen and mono-substituted, poly-substituted or unsubstituted, straight or branched chain variants of the following residues: C₁₋₆ alkyl, C₃₋₆ cycloalkyl, C₂₋₆ alkenyl, C₂₋₆ alkynyl, arylalkyl, alkylcarbonyl, C₂₋₆ alkoxycarbonyl, C₆₋₁₀ aryl, and C₅₋₁₀ heteroaryl.

In some embodiments, L is selected such that the piperizine nitrogen to which it is bound together with L form a tertiary amine, a carbamate, a urea, an amide, an enamine, a sulfamide, a sulfonamide, an aminal, a hydrazine, or a hydroxylamine. In some embodiments, L is selected from the group consisting of a carboxy group, a carbonyl group, and an alkoxy group. In some embodiments, L is selected from the group consisting of —C(O)O—, —C(O)—, and —(CH₂)₂O—.

In some embodiments, L is selected from a group consisting of —C(O)—, —C(O)O—, —C(O)NR¹⁰—, —NR¹⁰C(O)NR¹¹—, —NR¹⁰C(O)—, —NR¹⁰—, —CR¹⁰R¹¹OC(O)—, —CR¹⁰R¹¹C(O)O—,

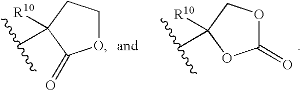

In some embodiments, Y is selected from a group consisting of hydrogen; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, phenyl-C₁₋₂-alkyl, C₃-C₂₄ heterocycloalkyl-alkyl, and C₃-C₂₄ heterocycloalkenyl-alkyl; and mono-substituted, poly-substituted or unsubstituted variants of the following residues: C₃-C₂₄ cycloalkyl, C₃-C₂₄ cycloalkenyl, C₂-C₂₄ cycloalkoxy, C₂-C₂₄ heterocycloalkyl, C₂-C₂₄ heterocycloalkenyl, acyl mono-radicals derived from naturally occurring amino acids or from lipoic acid, —CH₂CH₂SC(O)OR¹⁰, and —CH₂CH₂SC(O)R¹⁰.

In some embodiments, R² is H or F. In some embodiments, R⁸ is Cl, Br, or I. In some embodiments, R⁴ is Cl or Me. In some embodiments, X is NH. In some embodiments, X is O.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula R—C(O)O—R′, where R and R′ are independently selected from carbon-linked radicals.

An “amide” is a chemical moiety with formula R—C(O)NR′R″, where R, R′, and R″ are independently selected from carbon-linked radicals or hydrogen.

The term “tertiary amine” refers to a chemical moiety with formula NRR′R″, where R, R′, and R″ are independently selected from carbon-linked radicals.

The term “carbamate” refers to a chemical moiety with formula ROC(O)NR′R″, where R, R′, and R″ are independently selected from carbon-linked radicals.

The term “urea” refers to a chemical moiety with formula RR′NC(O)NR″R′″, where R, R′, R″, and R′″are independently selected from carbon-linked radicals.

The term “enamine” refers to a chemical moiety with formula RR′C═CR″NR′″, where R, R′, R′, and R′″ are independently selected from carbon-linked radicals of hydrogen.

The term “sulfamide” refers to a chemical moiety with formula RS(O)NR′R″, where R, R′, and R″ are independently selected from carbon-linked radicals or hydrogen.

The term “sulfonamide” refers to a chemical moiety with formula RS(O)₂NR′R″, where R, R′, and R″ are independently selected from carbon-linked radicals or hydrogen.

The term “aminal” refers to a chemical moiety with formula RD(OR′)(NR″)R′″, where R, R′, R″, and R′″ are independently selected from carbon-linked radicals or hydrogen.

The term “hydrazine” refers to a chemical moiety with formula RR′NNR″R′″, where R, R′, R″, and R′″ are independently selected from carbon-linked radicals or hydrogen.

The term “hydroxylamine” refers to a chemical moiety with formula RR′N—OR″, where R, R′, and R″ are independently selected from carbon-linked radicals or racicals or hydrogen.

The term “acetal” refers to a chemical moiety with formula RC(OR′)(OR″)R′″, where R, R′, R″, and R′″ are independently selected from carbon-linked radicals or hydrogen.

The term “carbonate” refers to a chemical moiety with formula ROC(O)OR′, where R and R′ are independently selected from carbon-linked radicals.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share, adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

The term “alkyl,” as used herein, means any unbranched or branched, substituted or unsubstituted, saturated hydrocarbon. The alkyl moiety, may be branched, straight chain, or cyclic. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

The term “perhaloalkyl” refers to an alkyl group where all of the hydrogen atoms are replaced by halogen atoms.

In the present context, the term “cycloalkyl” is intended to cover three-, four-, five-, six-, seven-, and eight- or more membered rings comprising carbon ring atoms only. Some examples of “cycloalkyl” are the carbocycles cyclopropane, cyclobutane, cyclopentane, cyclohexane, and cycloheptane.

An “alkenyl” moiety refers to a group consisting of at least two carbon atoms and at least one carbon-carbon double bond. An alkenyl may be unbranched or branched, substituted or unsubstituted, unsaturated hydrocarbon including polyunsaturated hydrocarbons. In some embodiments, the alkenyl is a C₁-C₆ unbranched, mono-unsaturated or di-unsaturated, unsubstituted hydrocarbons. The term “cycloalkenyl” refers to any non-aromatic hydrocarbon ring containing one or more carbon-carbon double bonds, preferably having five to twelve atoms comprising the ring. Non-limiting examples include cyclopentene, cyclopentadiene, cyclohexene, 1,3-cyclohexadiene, 1,4-cyclohexadiene, and cycloheptene.

An “alkyne” moiety refers to a group consisting of at least two carbon atoms and at least one carbon-carbon triple bond.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. The term “aryl” relates to aromatic, including, for example, benzenoid groups, connected via one of the ring-forming carbon atoms, and optionally carrying one or more substituents selected from heterocyclyl, heteroaryl, halo, hydroxy, amino, cyano, nitro, alkylamido, acyl, C₁₋₆ alkoxy, C₁₋₆ alkyl, C₁₋₆ hydroxyalkyl, C₁₋₆ aminoalkyl, C₁₋₆ alkylamino, alkylsulfenyl, alkylsulfinyl, alkylsulfonyl, sulfamoyl, or trifluoromethyl. The aryl group can be substituted at the para and/or meta positions. In other embodiments, the aryl group can be substituted at the ortho position. Representative examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl 3-cyanophenyl, 4-cyanophenyl, dimethylphenyl, naphthyl, hydroxynaphthyl, hydroxymethylphenyl, trifluoromethylphenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

In the present context, the term “heteroaryl” is intended to mean a heterocyclic aromatic group where one or more carbon atoms in an aromatic ring have been replaced with one or more heteroatoms (e.g., O, N, or S). Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole; indazole, tetrazole, quionoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

The term “alkoxy” refers to any unbranched, or branched, substituted or unsubstituted, saturated or unsaturated ether, with C₁-C₆ unbranched, saturated, unsubstituted ethers being preferred, with methoxy being preferred, and also with dimethyl, diethyl, methyl-isobutyl, and methyl-tert-butyl ethers also being preferred. The term “cycloalkoxy” refers to any non-aromatic hydrocarbon ring comprising an oxygen as a ring atom, preferably having five to twelve atoms comprising the ring.

An “O-carboxy” group refers to a RC(═O)O— group, where R is a carbon-linked radical or hydrogen.

A “C-carboxy” group refers to a —C(═O)OR groups where R is a carbon-linked radical or hydrogen.

An “acetyl” group refers to a —C(═O)CH₃ group.

A “trihalomethanesulfonyl” group refers to a X₃CS(═O)₂— group where X is a halogen.

A “cyano” group refers to a —CN group.

An “isocyanato” group refers to a —NCO group.

A “thiocyanato” group refers to a —CNS group.

An “isothiocyanato” group refers to a —NCS group.

A “sulfinyl” group refers to a —S(═O)—R group, where R is a carbon-linked radical or hydrogen.

A “S-sulfonamido” group refers to a —S(═O)₂NR group where R is a carbon-linked radical or hydrogen.

A “N-sulfonamido” group refers to a RS(═O)₂NH— group where R is a carbon-linked radical or hydrogen.

A “trihalomethanesulfonamido” group refers to a X₃CS(═O)₂NR— group with X is a halogen and R is a carbon-linked radical or hydrogen.

An “O-carbamyl” group refers to a —OC(═O)—NR group where R is a carbon-linked radical or hydrogen.

An “N-carbamyl” group refers to a ROC(═O)NH— group where R is a carbon-linked radical or hydrogen.

An “O-thiocarbamyl” group refers to a —OC(═S)—NR group where R is a carbon-linked radical or hydrogen.

An “N-thiocarbamyl” group refers to an ROC(═S)NH— group where R is a carbon-linked radical or hydrogen.

A “C-amido” group refers to a —C(═O)—NRR′ group where R and R′ are independently a carbon-linked radical or hydrogen.

An “N-amido” group refers to a RC(═O)NH— group where R is a carbon-linked radical or hydrogen.

The term “perhaloalkyl” refers to an alkyl group where all of the hydrogen atoms are replaced by halogen atoms.

The term “acylalkyl” refers to a RC(═O)R′— group where R is a carbon-linked radical or hydrogen and R′ is a diradical alkylene group. Examples of acylalkyl, without limitation, may include CH₃C(═O)CH₂—, CH₃C(═O)CH₂CH₂—, CH₃CH₂C(═O)CH₂CH₂—, CH₃C(═O)CH₂CH₂CH₂—, and the like.

The term “heterocyclyl” is intended to mean three-, four-, five-, six-, seven-, and eight- or more membered rings wherein carbon atoms together with from 1 to 3 heteroatoms constitute said ring. A heterocyclyl can optionally contain one or more unsaturated bonds situated in such a way, however, that an aromatic pi-electron system does not arise. The heteroatoms are independently selected from oxygen, sulfur, and nitrogen.

A heterocyclyl can further contain one or more carbonyl or thiocarbonyl functionalities, so as to make the definition include oxo-systems and thio-systems such as lactams, lactones, cyclic imides, cyclic thioimides, cyclic carbamates, and the like.

Heterocyclyl rings can optionally also be fused to aryl rings, such that the definition includes bicyclic structures. Typically such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Binding to the heterocycle can be at the position of a heteroatom or via a carbon atom of the heterocycle, or, for benzo-fused derivatives, via a carbon of the benzenoid ring.

Unless otherwise indicated, when a substituent is deemed to be “optionally subsituted,” it is meant that the subsitutent is a group that may be substituted with one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, Protective Groups in Organic Synthesis, 3^{rd} Ed., John Wiley & Sons, New York, N.Y., 1999, which is incorporated herein in its entirety.

In some embodiments, the compounds described herein may be capable of being metabolically transformed into a compound capable of modulating the activity of a muscarinic receptor.

The term “modulate” refers to the ability of a compound disclosed herein to alter the function of a muscarinic receptor. A modulator may activate the activity of a muscarinic receptor, may activate or inhibit the activity of a muscarinic receptor depending on the concentration of the compound exposed to the muscarinic receptor, or may inhibit the activity of a muscarinic receptor. The term “modulate” also refers to altering the function of a muscarinic receptor by increasing or decreasing the probability that a complex forms between a muscarinic receptor and a natural binding partner. A modulator may increase the probability that such a complex forms between the muscarinic receptor and the natural binding partner, may increase or decrease the probability that a complex forms between the muscarinic receptor and the natural binding partner depending on the concentration of the compound exposed to the muscarinic receptor, and or may decrease the probability that a complex forms between the muscarinic receptor and the natural binding partner. In some embodiments, modulation of the muscarinic receptor may be assessed using Receptor Selection and Amplification Technology (R-SAT) as described in U.S. Pat. No. 5,707,798, the disclosure of which is incorporated herein by reference in its entirety.

The term “activate” refers to increasing the cellular function of a muscarinic receptor. The term “inhibit” refers to decreasing the cellular function of a muscarinic receptor. The muscarinic receptor function may be the interaction with a natural binding partner or catalytic activity.

The term “contacting” as used herein refers to bringing a compound disclosed herein and a target muscarinic receptor together in such a manner that the compound can affect the activity of the muscarinic receptor, either directly; i.e., by interacting with the muscarinic receptor itself, or indirectly; i.e., by interacting with another molecule on which the activity of the muscarinic receptor is dependent. Such “contacting” can be accomplished in vitro in a test tube, a petri dish or the like. In vitro, contacting may involve only a compound and a muscarinic receptor of interest or it may involve whole cells. Cells may also be maintained or grown in cell culture dishes and contacted with a compound in that environment. In this context, the ability of a particular compound to affect a muscarinic receptor related disorder; i.e., the IC₅₀ of the compound can be determined before use of the compounds in vivo with more complex living organisms is attempted. For cells outside the organism, multiple methods exist, and are well-known to those skilled in the art, to get the muscarinic receptors in contact with the compounds including, but not limited to, direct cell microinjection and numerous transmembrane carrier techniques. The term “contacting” can also refer to bringing a compound disclosed herein to contact with a target muscarinic receptor in vivo. Thus, if a compound disclosed herein, or a prodrug thereof, is administered to an organism and the compound is brought together with a muscarinic receptor within the organism, such contacting is within the scope of the present disclosure.

In some embodiments, the pharmacologically active compound formed from the prodrug compound of Formula I or II may be an agonist of said receptor, while in other embodiments, the active compound may be an antagonist of said receptor. In yet other embodiments, the compound may be a partial agonist of said receptor. A compound that is a partial agonist may in some cases be a partial activator of a receptor, while in other cases may be a partial repressor of a receptor. In yet other circumstances, the compound may be a tissue-specific modulator, while in other circumstances, the compound may be a gene-specific modulator.

In some embodiments, compounds according to Formula I or II may be administered to a patient to treat a condition associated with a muscarinic receptor. In one embodiment, the compound is administered to treat or prevent a neuropsychiatric disorder. In some embodiments, the neuropsychiatric disorder is selected from the group consisting of one or more of psychosis of any origin, cognitive impairment associated with psychosis, hallucinations, delusions, disordered thought, behavioral disturbance, aggression, neuropathic pain, anhedonia, psychiatric and other behavioral disturbances characteristic of dementia or cognitive impairment of any origin, schizophrenia and related idiopathic psychoses, anxiety, sleep disorders, appetite disorders, affective disorders such as major depression, bipolar disorder, mania, flattening of affect, suicidality, and depression with psychotic features, Tourette's Syndrome, drug-induced psychoses, and symptoms such as psychosis and cognitive impairment associated with neurodegenerative disorders such as Alzheimer's or Huntington's Disease. In one embodiment, the compound is administered to treat glaucoma. In some embodiments, prior to administration of a compound according to Formula I or II, the patient is first identified as suffering from one or more of the above-described conditions.

Certain of the compounds disclosed herein may exist as stereoisomers including optical isomers. The scope of the present disclosure includes all stereoisomers and both the racemic mixtures of such stereoisomers as well as the individual enantiomers that may be separated according to methods that are well known to those of ordinary skill in the art.

In another aspect, the present disclosure relates to a pharmaceutical composition comprising a physiologically acceptable carrier, diluent, or excipient, or a combination thereof, and a compound of Formula I or II.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethycellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

In some embodiments, the present disclosure relates to a pharmaceutical composition comprising a compound of Formula I or II and an additional neuropsychiatric agent. As used herein, a “neuropsychiatric agent” refers to a compound, or a combination of compounds, that affects the neurons in the brain either directly or indirectly, or affects the signal transmitted to the neurons in the brain. Neuropsychiatric agents, therefore, may affect a person's psyche, such as the person's mood, perception, nociception, cognition, alertness, memory, etc. In certain embodiments, the neuropsychiatric agent may be selected from the group consisting of a selective serotonin reuptake inhibitor, norepinephrine reuptake inhibitor, dopamine agonist, antipsychotic agent, serotonin 2A antagonists, and inverse serotonin 2A agonists.

In some embodiments, the antipsychotic agent may be selected from the group consisting of a phenothiazine, phenylbutylpiperadine, debenzapine, benzisoxidil, and salt of lithium. The phenothiazine group of compounds may be selected from the group consisting of chlorpromazine (Thorazine®), mesoridazine (Serentil®), prochlorperazine (Compazine®), and thioridazine (Mellaril®). The phenylbutylpiperadine group of compounds may be selected from the group consisting of haloperidol (Haldol®), and pimozide (Orap®). The debenzapine group of compounds may be selected from the group consisting of clozapine (Clozaril®), loxapine (Loxitane®), olanzapine (Zyprexa®) and quetiapine (Seroquel®). The benzisoxidil group of compounds may be selected from the group consisting of resperidone (Resperidal®) and ziprasidone (Geodon®). The salt of lithium may be lithium carbonate. In some embodiments, the antipsychotic agent may be selected from the group consisting of Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, and Zyprexa, or pharmaceutically acceptable salts thereof.

In certain embodiments, the selective serotonin reuptake inhibitor is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

In other embodiments, the norepinephrine reuptake inhibitor is selected from the group consisting of thionisoxetine and reboxetine.

In further embodiments, the dopamine agonist is selected from the group consisting of sumatriptan, almotriptan, naratriptan, frovatriptan, rizatriptan, zomitriptan, cabergoline, amantadine, lisuride, pergolide, ropinirole, pramipexole, and bromocriptine.

In another embodiment, the inverse serotonin 2A agonist is a compound of Formula III:

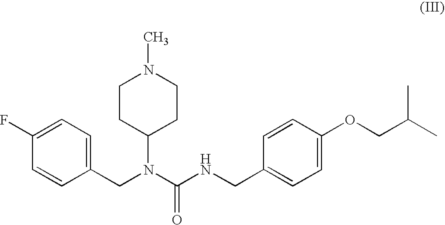

In another embodiment, the serotonin 2A antagonist is M 100,907 or an analog thereof. By “M 100,907,” it is meant the compound of Formula IV.

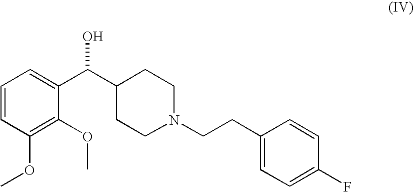

Another embodiment includes a method of treating a neuropsychiatric disorder in a patient comprising administering to the patient a therapeutically effective amount of a pharmaceutical composition comprising a compound of Formula I or II and a neuropsychiatric agent. Another embodiment includes a method of treating neuropsychiatric disorder in a patient comprising administering to the patient a therapeutically effective amount of a compound of Formula I or II and a therapeutically effective amount of a neuropsychiatric agent.

In some embodiments, the administering step in the above methods comprises administering the compound of Formula I or II and the neuropsychiatric agent nearly simultaneously. These embodiments include those in which the compound of Formula I or II and the neuropsychiatric agent are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments, after administration of one of the agents, the additional agent is administered at some latter time (e.g., a few minutes or a few hours latter). Also included in these embodiments are those in which the patient is administered a composition comprising one of the compounds on a routine or continuous basis while receiving a composition comprising the other compound occasionally.

### EXAMPLES

General Procedure 1 (GP1)

The acid chloride or the chloroformate form of the desired compound (0.5 mmol) was added to a solution of 8-chloro-11-(1-piperazinyl)-5H-dibenzo[be]-[1,4]diazepine (0.5 mmol, 0.157 g) and triethylamine (0.22 mL, 1.5 mmol) in dry CH₂Cl₂ (5 ml) at 0° C. The reaction mixture was stirred at room temperature for 15 hours. The reaction was quenched by addition of saturated aqueous NaHCO₃ (5 ml) and the organic phase was washed with brine (5 ml), dried (Na₂SO₄), filtered and concentrated at reduced pressure. The crude product was purified by preparative TLC (ethyl acetate:n-heptane, 1:4).

General Procedure 2 (GP2)

The acid chloride or the chloroformate form of the desired compound (1.2 mmol) was added to a solution of 8-chloro-11-(1-piperazinyl)-5H-dibenzo[b,e]-[1,4]diazepine (313 mg, 1.0 mmol) and triethylamine (0.22 mL, 1.5 mmol) in CH₂Cl₂ (10 mL) at −70° C. The reaction mixture was allowed to obtain room temperature over night and was then diluted with CH₂Cl₂, washed with saturated aqueous NaHCO₃, dried (Na₂SO₄), and concentrated at reduced pressure. The crude product was purified by column chromatography (SiO₂, acetone:MeOH systems).

### Example 1

### 4-(8-Chloro-5H-dibenzo[b,e][1,4]diazepin-11-yl)-piperazine-1-carboxylic acid 2,2,2-trichloroethyl ester (212GG95-3)

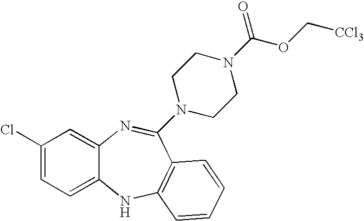

8-Chloro-11-(1-piperazinyl)-5H-dibenzo[be]-[1,4]diazepine and 2,2,2-trichloroethyl chloroformate were reacted according to GP1 to give 8.2 mg of the title compound 212GG95-3. MS (ESI) 487 (MH⁺). Purity for MH⁺ (UV/MS) 95/90.

### Example 2

### 4-(8-Chloro-5H-dibenzo[b,e][1,4]diazepin-11-yl)-piperazine-1-carboxylic acid dodecyl ester (212GG96)

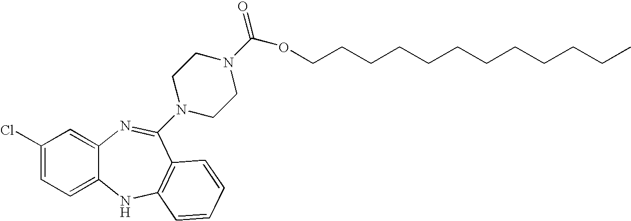

8-Chloro-11-(1-piperazinyl)-5H-dibenzo[b,e]-[1,4]diazepine and dodecyl chloroformate were reacted according to GP1 to give 105.5 mg of the title compound 212GG96. MS (ESI) 525 (MH⁺). Purity for MH⁺ (UV/MS) 95/90.

### Example 3

### 4-(8-Chloro-5H-dibenzo[b,e][1,4]diazepin-11-yl)-piperazine-1-carboxylic acid ethyl ester (240GG01)

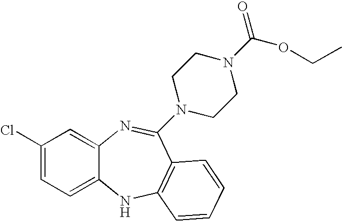

8-Chloro-11-(1-piperazinyl)-5H-dibenzo[b,e]-[1,4]diazepine and ethyl chloroformate were reacted according to GP1 to give 75.0 mg of the title compound 240GG01. MS (ESI) 385 (MH⁺). Purity for MH⁺ (UV/MS) 100/92.

### Example 4

### 4-(8-Chloro-5H-dibenzo[b,e][1,4 diazepin-11-yl)-piperazine-1-carboxylic acid phenyl ester (212GG95-3)

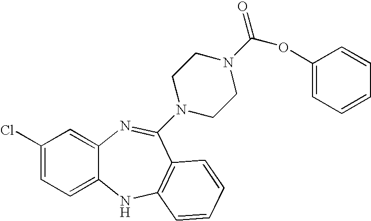

8-Chloro-11-(1-piperazinyl)-5H-dibenzo[b,e]-[1,4]diazepine and phenyl chloroformate were reacted according to GP1 to give 60.2 mg of the title compound 212GG95-3. MS (ESI) 433 (MH⁺). Purity for MH⁺ (UV/MS) 100/83.

### Example 5

### 4-(8-Chloro-5H-dibenzo[b,e][1,4]diazepin-11-yl)-piperazine-1-carboxylic acid allyl ester (240GG16-1)

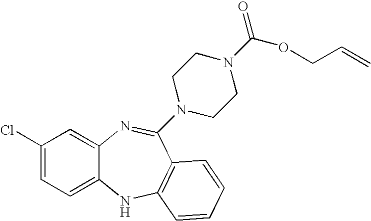

8-Chloro-11-(1-piperazinyl)-5H-dibenzo[b,e]-[1,4]diazepine and allyl chloroformate were reacted according to GP1 to give 30.0 mg of the title compound 240GG16-1. MS (ESI) 397 (MH⁺). Purity for MH⁺ (UV/MS) 100/70.

### Example 6

### 4-(8-Chloro-5H-dibenzo[b,e][1,4]diazepin-11-yl)-piperazine-1-carboxylic acid isobutyl ester (240GG16-2)

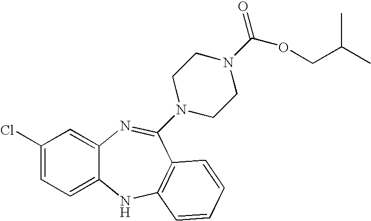

8-Chloro-11-(1-piperazinyl)-5H-dibenzo[b,e]-[1,4]diazepine and isobutyl chloroformate were reacted according to GP1 to give 120.2 mg of the title compound 240GG16-2. MS (ESI) 413 (MH⁺). Purity for MH⁺ (UV/MS) 100/73.

### Example 7

### 4-(8-Chloro-5H-dibenzo[b,e][1,4]diazepin-11-yl)-piperazine-1-carboxylic acid 2-chloroethyl ester (240GG16-3)

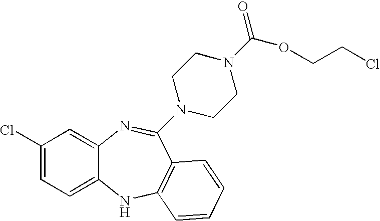

8-Chloro-11-(1-piperazinyl)-5H-dibenzo[b,e]-[1,4]diazepine and 2-chloroethyl chloroformate were reacted according to GP1 to give 60.2 mg of the title compound 240GG16-3. MS (ESI) 419 (MH⁺). Purity for MH⁺ (UV/MS) 100/75.

### Example 8

### 4-(8-Chloro-5H-dibenzo[b,e][1,4]diazepin-11-yl)-piperazine-1-carboxylic acid octyl ester (240GG16-4)

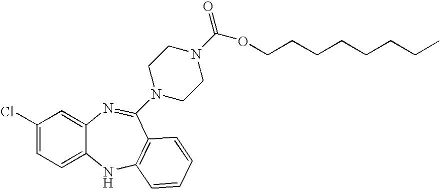

8-Chloro-11-(1-piperazinyl)-5H-dibenzo[b,e]-[1,4]diazepine and octyl chloroformate were reacted according to GP1 to give 70.4 mg of the title compound 240GG16-4. MS (ESI) 469 (MH⁺). Purity for MH⁺ (UV/MS) 100/80.

### Example 9

### 4-(8-Chloro-5H-dibenzo[b,e][1,4]diazepin-11-yl)-piperazine-1-carboxylic acid hexadecyl ester (240GG16-5)

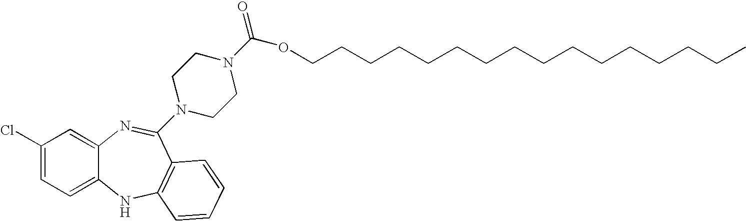

8-Chloro-11-(1-piperazinyl)-5H-dibenzo[b,e]-[1,4]diazepine and hexadecyl chloroformate were reacted according to GP1 to give 62.7 mg of the title compound 240GG16-5. Purity for MH⁺ (UV/MS) NA (compound too hydrophobic for HPLC/MS).

### Example 10

### 4-(8-Chloro-5H-dibenzo[b,e][1,4]diazepin-11-yl)-piperazine-1-carboxylic acid 4-nitrobenzyl ester (240GG09-5)

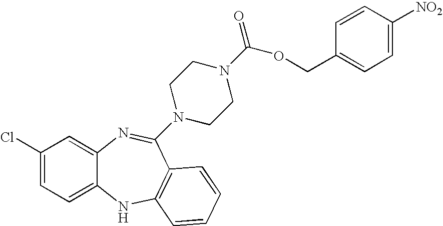

8-Chloro-11-(1-piperazinyl)-5H-dibenzo[b,e]-[1,4]diazepine and 4-nitrobenzyl chloroformate were reacted according to GP1 to give 62.7 mg of the title compound 240GG09-5. MS (ESI) 492 (MH⁺). Purity for MH⁺ (UV/MS) 100/63.

### Example 11

### 4-(8-Chloro-5H-dibenzo[b,e][1,4]diazepin-11-yl)-piperazine-1-carboxylic acid tert-butyl ester (222JO83)

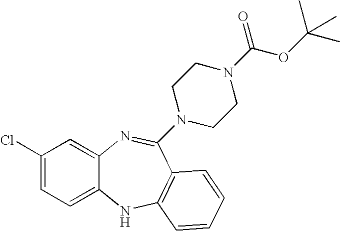

8-Chloro-11-(1-piperazinyl)-5H-dibenzo[b,e]-[1,4]diazepine (200 mg, 0.64 mmol) and di-t-butyldicarbonate (168 mg, 0.77 mmol) were reacted using the procedure described for GP2 to give 120 mg of the title compound (222JO83). MS (ESI) 413 (MH⁺). Purity for MH⁺ (UV/MS) 100/100

### Example 12

### 4-(8-Chloro-5H-dibenzo[b,e][1,4]diazepin-11-yl)-piperazine-1-carboxylic acid methyl ester-one (222JO65)

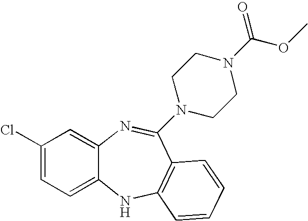

8-Chloro-11-(1-piperazinyl)-5H-dibenzo[b,e]-[1,4]diazepine (200 mg, 0.64 mmol) and methyl chloroformate (0.069 mL, 0.9 mmol) were reacted according to GP2 to give 155 mg of the title compound (222JO65). MS (ESI) 371 (MH⁺). Purity for M⁺ (UV/MS) 99/92.

### Example 13

### 1-[4-(8-Chloro-5H-dibenzo[b,e][1,4]diazepin-11-yl)-piperazin-1-yl]-butan-1-one (222JO91)

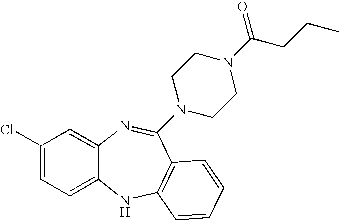

8-Chloro-11-(1-piperazinyl)-5H-dibenzo[b,e]-[1,4]diazepine (313 mg, 1.0 mmol) and buturyl chloride (128 mg, 1.2 mmol) were reacted according to GP2 to give 210 mg of the title compound (222JO91). MS (ESI) 383 (MH⁺). Purity for MH⁺ (UV/MS) 100/100.

### Example 14

### 1-[4-(8-Chloro-5H-dibenzo[b,e][1,4]diazepin-11-yl)-piperazin-1-yl]-octan-1-one (222JO93a)

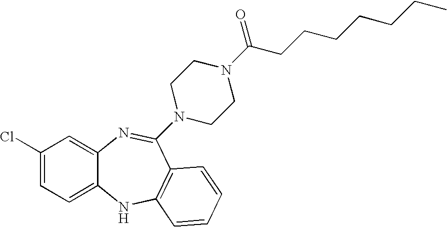

8-Chloro-11-(1-piperazinyl)-5H-dibenzo[b,e]-[1,4]diazepine (157 mg, 1.0 mmol) and octanoyl chloride (98 mg, 0.6 mmol) were reacted according to GP2 to give 173 mg of the title compound (222JO93a). MS (ESI) 439 (MH⁺). Purity for MH⁺ (UV/MS) 100/100.

### Example 15

### 1-[4-(8-Chloro-5H-dibenzo[b,e][1,4]diazepin-11-yl)-piperazin-1-yl]-dodecan-1-one (222JO93b)

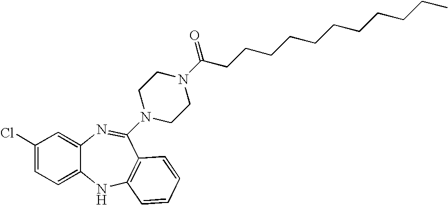

8-Chloro-11-(1-piperazinyl)-5H-dibenzo[b,e]-[1,4]diazepine (157 mg, 1.0 mmol) and lauoryl chloride (131 mg, 0.6 mmol) were reacted according to GP2 to give 204 mg of the title compound (222JO93b). MS (ESI) 495 (MH⁺). Purity for MH⁺ (UV/MS) 100/100.

### Example 16

### 1-[4-(8-Chloro-5H-dibenzo[b,e][1,4]diazepin-11-yl)-piperazin-1-yl-hexadecan-1-one (222JO93c)

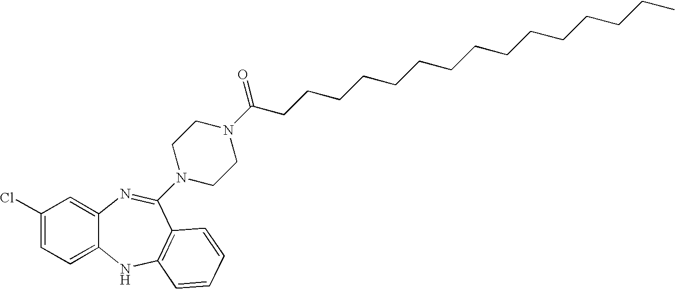

8-Chloro-11-(1-piperazinyl)-5H-dibenzo[b,e]-[1,4]diazepine (157 mg, 1.0 mmol) and palmitoyl chloride (165 mg, 0.6 mmol) were reacted according to GP2 to give 237 mg of the title compound (222JO93c). MS (ESI) 551 (MH⁺). Purity for MH⁺ (UV/MS) 100/100.

### Example 17

### 1-[4-(8-Chloro-5H-dibenzo[b,e][1,4]diazepin-11-yl)-piperazin-1-yl]-etan-1-one (222JO94a)

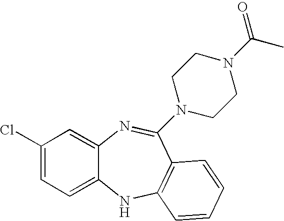

Acetic anhydride (0.057 mL, 0.6 mmol) was added to a solution of 8-chloro-11-(1-piperazinyl)-5H-dibenzo[b,e]-[1,4]diazepine (157 mg, 0.5 mmol) in CH₂Cl₂ (3 mL) at room temperature. The reaction mixture was stirred at room temperature for 1 h, then concentrated to about half the volume and purified by column chromatography (SiO₂, heptane:EtAOAc:MeOH) to give 152 mg of the title compound (222JO94a). MS (ESI) 355 (MH⁺). Purity for MH⁺ (UV/MS) 100/100.

### Example 18

### 1-[4-(8-Chloro-5H-dibenzo[b,e][1,4]diazepin-11-yl)-piperazin-1-yl]-propan-1-one (222JO94b)

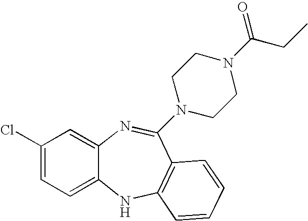

8-Chloro-11-(1-piperazinyl)-5H-dibenzo[b,e]-[1,4]diazepine (157 mg, 1.0 mmol) and propionic anhydride (0.077 mL, 0.6 mmol) were reacted as described for Example 17 to give 155 mg of the title compound (222JO94b). MS (ESI) 369 (MH⁺). Purity for MH⁺ (UV/MS) 100/100.

### Example 19

### 4-(8-Chloro-5H-dibenzo[b,e][1,4]diazepin-11-yl)-piperazine-1-carbaldehyde (222JO95)

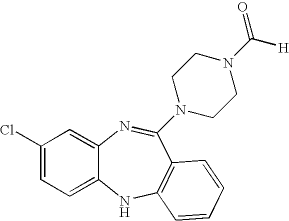

Ethyl formate (0.40 mL, 5.0 mmol) and triethylamine (0.14 mL, 1.0 mmol) were added to a solution of 8-chloro-11-(1-piperazinyl)-5H-dibenzo[b,e]-[1,4]diazepine (157 mg, 0.5 mmol) in dioxane (3 ml). The reaction mixture was stirred at 50° C. for 16 h, then at 80° C. for 5h and was then concentrated at reduced pressure. The residue was purified by column chromatography (SiO₂, heptane:EtOAc, 1:1, then CH₂Cl₂:MeOH, 10:1) to give 124 mg of the title compound (222JO95). MS (ESI) 341 (MH⁺). Purity for MH⁺ (UV/MS) 100/100.

### Example 20

### Acetic acid 2-{2-[4-(8-chloro-5H-dibenzo[b,e][1,4]diazepin-11-yl)-piperazin-1-yl]-ethoxy}-ethyl ester (247JO03A)

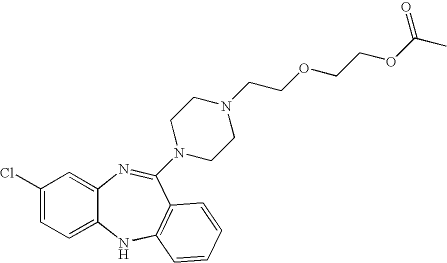

Di-t-butyldicarbonate (7 mmol) was added to a solution of 1-(2-(2-hydroxyethoxy)ethyl)piperazine (870 mg, 5 mmol) in CH₂Cl₂ (20 mL). The reaction mixture was stirred at room temperature over night and was then diluted with CH₂Cl₂, washed with saturated aqueous NaHCO₃-solution, dried (Na₂SO₄) and concentrated. The residue was dissolved in CH₂Cl₂ (20 mL) and acetic anhydride (1.18 mL, 12.5 mmol) and pyridine (1.20 mL, 15 mmol) were added. The resulting reaction mixture was stirred over night at room temperature, then diluted with CH₂Cl₂, washed with saturated aqueous NaHCO₃-solution, dried (Na₂SO₄), and concentrated at reduced pressure. The residue was purified by column chromatography (toluene:acetone, 5:1, 3:1) to give 1.41 g of 4-[2-(2-acetoxyethoxy)ethyl]-piperazine-1-carboxylic acid tert-butyl ester (247JO01). ¹H NMR (CDCl₃) δ 4.22 (t, 2 H, J=6.9 Hz), 3.66 (m 4 H,), 3.45 (m, 4 H), 2.66 (m, 2 H), 2.48 (m, 4 H), 2.07 (s, 3 H), 1.46 (s, 9H).

Trifluoroacetic acid (0.5 mL) was added to a solution of 4-[2-(2-acetoxyethoxy)ethyl]-piperazine-1-carboxylic acid tert-butyl ester (247JO01) (100 mg, 0.32 mmol) in CH₂Cl₂ (1 mL). The reaction mixture was stirred at room temperature for 1 h and was then diluted with CH₂Cl₂ and washed with a 2 M NaOH aqueous solution saturated with NaCl. The aqueous phase was extracted with CH₂Cl₂ (3×). The combined organic phases were dried (Na₂SO₄) and concentrated at reduced pressure.

The residue was dissolved in dioxane (3 mL) and 8,5-dichloro-5H-dibenzo[b,e][1,4]diazepine (56 mg, 0.17 mmol) and triethylamine (0.029 mL, 0.20 mmol) were added. The resulting reaction mixture was stirred at 80° C. for 16 h and was then diluted with CH₂Cl₂, washed with saturated aqueous NaHCO₃-solution, dried (Na₂SO₄), and concentrated at reduced pressure. The residue was purified by column chromatography (SiO₂, toluene:acetone:MeOH: 4:1:0, 1:1:0.05) to give 13 mg of the title compound (247JO03A), MS (ESI) 443 (MH⁺), Purity for MH⁺ (UV/MS) 96/100.

### Example 21

### 2-{2-[4-(8-Chloro-5H-dibenzo[b,e][1,4]diazepin-11-yl)-piperazin-1-yl]-ethoxy}-ethanol (247J003B)

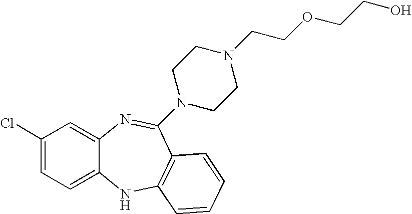

The procedure described in Example 20 provided 12 mg of the title compound (247JO03B), MS (ESI) 401 (MH⁺). Purity for MH⁺ (UV/MS) 99/78

### Example 22

### 4-(8-Chloro-dibenzo[b,f][1,4]oxazepin-11-yl)-piperazine-1-carboxylic acid tert-butyl ester (222JO05A)

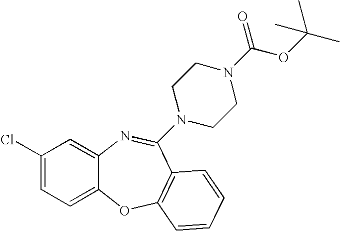

8-Chloro-10H-dibenzo[b,f][1,4]oxazepin-11-one (47.5 mg, 1.93 mmol) was dissolved in neat POCl₃ (10 ml) and the reaction mixture was stirred at 100° C. for 15 hours. The reaction mixture was concentrated at reduced pressure and the residue was dissolved in dichloromethane (10 ml) and washed with saturated aqueous NaHCO₃-solution (2×10 nm). The organic phase was dried (Na₂SO₄), filtered, and concentrated at reduced pressure to give a crude product (60.0 mg) that was used without further purifications.

t-Butyl-1-piperazinecarboxylate (29 mg, 0.16 mmol) and triethylamine (0.023 mL., 0.16 mmol) were added to a solution of the crude 8,5-dichloro-dibenzo[b,f][1,4]oxazepine (30 mg) in dioxane. The reaction mixture was stirred over night at 80° C. and then at 110° C. for 3 h. After cooling, the mixture was diluted with CH₂Cl₂, washed with saturated aqueous NaHCO₃-solution, dried (Na₂SO₄), and concentrated at reduced pressure. The residue was purified by column chromatography (SiO₂, heptane:EtOAc 4:1) to give crude product. Further purification (SiO₂, toluene:acetone, 10:1) gave 10.9 mg of the title compound (247JO05A), MS (ESI) 414 (MH⁺), Purity for MH⁺ (UV/MS) 91/98.

### Example 23

### 1-[4-(8-Chloro-dibenzo[b,f][1,4]oxazepin-11-yl)-piperazin-1-yl]-ethanone (247JO05B)

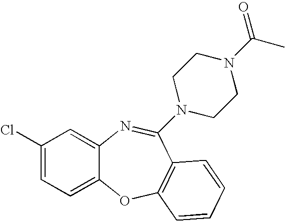

1-Acetylpiperazine (20 mg, 0.16 mmol) and triethylamine (0.023 mL, 0.16 mmol) were added to a solution of crude 8,5-dichloro-dibenzo[b,f][1,4]oxazepine (30 mg, Example 22) in dioxane and the reaction mixture was stirred over night at 80° C. and then at 110° C. for 3 h. After cooling, the reaction mixture was diluted with CH₂Cl₂, washed with saturated aqueous NaHCO₃-solution, dried (Na₂SO₄), and concentrated at reduced pressure. The residue was purified by column chromatography (SiO₂, toluene:acetone, 4:1, 2:1) to give 4.9 mg of the title compound (247JO05B), MS (ESI) 356 (MH⁺), Purity for MH⁺ (UV/MS) 98/92.

### Example 24

### (2S)-{1-[4-(8-Chloro-5H-dibenzo[b,e][1,4]diazepin-11-yl)-piperazine-1-carbonyl]-3-methyl-butyl}-carbamic acid tert-butyl ester (240GG28-2)

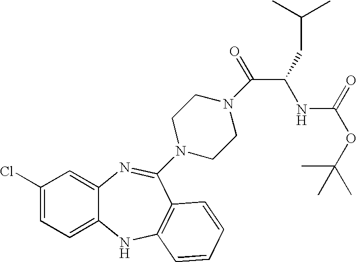

8-Chloro-11-(1-piperazinyl)-5H-dibenzo[b,e]-[1,4]diazepine (0.16 g, 0.5 mmol) and Boc-L-leucine (0.5 mmol) were dissolved in dry dichloromethane (5 ml). EDC (0.11 g, 0.55 mmol) was added at room temperature and reaction was stirred at room temperature for 4 hours.

Water (5 ml) was added and the organic phase was separated then washed with brine (10 ml). The organic layer was dried over sodium sulfate, filtered and evaporated to give a crude oil which was purified by silica gel flash chromatography (eluent: ethyl acetate/heptanes 1/1) to give 140.3 mg of the title compound. Purity for MH+ (526) (UV/MS) 100/91.

### Example 25

### (2S)-2-Amino-1-[4-(8-chloro-5H-dibenzo[b,e][1,4]diazepin-11-1-yl]-4-methyl-pentan-1-one (240GG29-2)

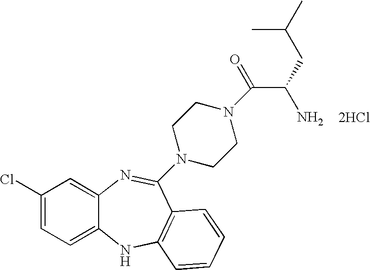

(2S)-{1-[4-(8-Chloro-5H-dibenzo[b,e][1,4]diazepin-11-yl)-piperazine-1-carbonyl]-3-methyl-butyl}-carbamic acid tert-butyl ester (120.0 mg) was treated with a solution of HCl in dioxane (4N, 15 ml) for 3 hours at room temperature. The product was filtered off the reaction mixture then washed with diethyl ether to give the title compound as a white solid (95.0 mg, hydrochloride salt). Purity for MH+ (426) (UV/MS) 100/100.

### Example 26

### [4-(8-Chloro-5H-dibenzo[b,e][1,4]diazepin-11-yl)-piperazin-1-yl]-pyridin-3-yl-methanone (240GG71)

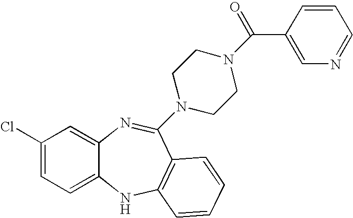

8-Chloro-11-(1-piperazinyl)-5H-dibenzo[b,e]-[1,4]diazepine (1 g, 3.2 mmol) was co-evaporated with dry pyridine (2×10 ml), and then dissolved in dry pyridine (20 ml). Nicotinyl chloride hydrochloride (0.57 g, 3.2 mmol) was added at 0° C. then the reaction was stirred at room temperature for 15 hours.

The reaction was evaporated under vacuum, dissolved in AcOEt (100 ml) and washed with a saturated aqueous solution of sodium bicarbonate (2×10 ml). The organic layer was dried over sodium sulfate, filtered and evaporated to give the title compound (2.20 g) as a clear oil used without further purifications. Purity for MH+ (417) (UV/MS) 92/86.

### Example 27

### [4-(8-Chloro-5H-dibenzo[b,e][1,4]diazepin-11-yl)-piperazin-1-yl]-(1-methyl-pyridin-3-yl)-methanone iodide (240GG72)

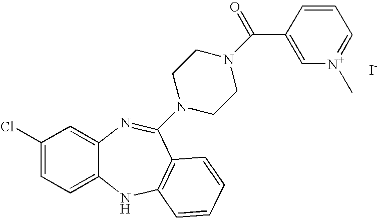

[4-(8-Chloro-5H-dibenzo[b,e][1,4]diazepin-11-yl)-piperazin-1-yl]-pyridin-3-yl-methanone (2.20 g, 5.26 mmol) was dissolved in methanol (50 ml). Methyl iodide (0.33 ml, 10.52 mmol) was added at room temperature and the reaction was refluxed for 15 hours. The reaction was evaporated to dryness to give the title compound as a foam and was used without further purifications.

### Example 28

### [4-(8-Chloro-5H-dibenzo[b,e][1,4]diazepin-11-yl)-piperazin-1-yl-(1-methyl-1,4-dihydro-pyridin-3-yl)-methanone (240GG73)

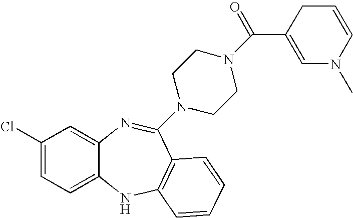

[4-(8-Chloro-5H-dibenzo[b,e][1,4]diazepin-11-yl)-piperazin-1-yl]-(1-methyl-pyridin-3-yl)-methanone iodide (2.00 g, 3.57 mmol) is dissolved in ethanol/water (1/1 v/v, 50 ml). Sodium bicarbonate (1.78 g, 21.43 mmol) and sodium hydrosulfite (2.49 g, 14.29 mmol) were added at room temperature. The reaction was stirred at room temperature for 15 hours then concentrated under vacuum. The residue was extracted with AcOEt (2×100 ml): The organic layer was washed with brine (100 ml), dried over sodium sulfate, filtered and evaporated to give a crude solid. A portion of this crude material (0.10 g) was purified by preparative thin layer chromatography (eluent: 5% MeOH/dichloromethane) to give the title compound (45.0 mg). Purity for MH+ (434) (UV/MS) 100/100.

### Example 29

### 2-[4-(8-Chloro-5H-dibenzo[b,e][1,41diazepin-11-yl)-piperazin-1-yl]-ethanol (240GG43)

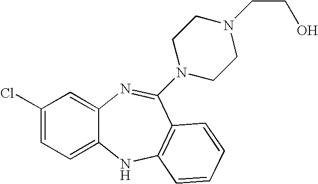

8-Chloro-11-(1-piperazinyl)-5H-dibenzo[b,e]-[1,4]diazepine (0.30 g, 0.96 mmol) was dissolved in dry acetonitrile (10 ml). Iodoethanol (0.15 ml, 1.92 mmol) and cesium carbonate (0.62 g, 1.92 mmol) were added at room temperature and the reaction was stirred at 50° C. for 15 hours. The reaction was evaporated with celite and purified by silica gel chromatography (eluent:20% MeOH/dichloromethane) to give the title compound (0.36 g). Purity for MH+ (357) (UV/MS) 96/80.

### Example 30

### 4-(8-Chloro-5H-dibenzo[b,e][1,4]diazepin-11-yl)-piperazine-1-carboxylic acid 5-methyl-2-oxo-[1,3]dioxol-4-yl methyl ester (240GG36)

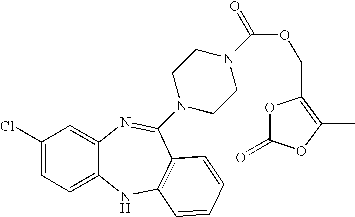

8-Chloro-11-(1-piperazinyl)-5H-dibenzo[b,e]-[1,4]diazepine (0.11 g, 0.34 mmol) was dissolved in dry DMF (5 ml). Carbonic acid 5-methyl-2-oxo-[1,3]dioxol-4-ylmethyl ester 4-nitro-phenyl ester (0.10 g, 0.34 mmol) was added and the reaction was stirred for 16 hours at room temperature. Water was added (50 ml) and the reaction was extracted with AcOEt (2×25 ml). The organic layer was dried over sodium sulfate, filtered and evaporated to give a crude solid. The compound was purified by silica gel column chromatography (AcOEt/n-heptane 1/1) to afford the title compound (67.0 mg). Purity for MH+ (469) (UV/MS) 94/80.

### Example 31

### Conversion of Prodrugs to NDMC In Vivo

Male, Sprauge-Dawly rats (200-225 grams, Harlan, San Diego, Calif.) implanted with jugular vein cannulae served as experimental subjects. Rats were fasted for approximately 16 hours and then dosed orally with one of several test compounds. All test compounds were administered at a fixed dose of 10 mg/kg (freebase) in a volume of 1 mL/kg. Venous blood samples were collected 0, 30, 60 and 120 min following test compound administration. Samples were placed into heparinized tubes and centrifuged to obtain plasma. Additionally, whole brains were harvested at the 120 min time point in order to determine brain levels of test compounds. Plasma samples, brains and any remaining dosing solutions were stored at −80° C. until analyzed.

The thawed plasma samples were processed by protein precipitation using acetonitrile. Following centrifugation the supernatant was diluted appropriately and transferred to the analysis deep well plate for analysis.

Calibration curves were constructed for each analyte by spiking of blank plasma. An identical procedure was used for processing of calibration samples and plasma samples. The final range of the calibration curve was 2-1250 nM.

Calibration samples and samples were analyzed by LC-MS/MS using positive electrospray ionization. The analytical system consisted of a Waters® 1525μ binary HPLC pump with Micromass Quattro Ultima™ Pt MS/MS system and a CTC HTC PAL autosampler. Data collection and processing was performed using MassLynx software v 4.0.

The sample (10 μL) was injected on the column (Phenomenex Synergi Fusion RP, 50×2.00 mm 4 μm particles). The compounds were eluted from the column using a solvent gradient at a flow rate of 0.800 mL/min. The composition of the mobile phase was the following: A: 95% purified water, 5% methanol +0.5% acetic acid and B: 95% methanol, 5% purified water+0.5% acetic acid. The initial condition of 0% B was gradually ramped to 45% B in 1.00 min. The solvent mixture was then changed to 55% B in 1.60 min followed by 100% B in 0.6 min. The condition of 100% B was maintained in 0.6 min. The gradient was then returned to 0% B in 0.2 min and maintained in 1.5 min for equilibration of the column. The total runtime was 5.5 min.

Samples were analyzed using multiple reaction monitoring (MRM). MS settings were defined by Quanoptimize to give maximum abundance of parent and daughter ions.

Plasma concentrations versus time data were subjected to non-compartmental analysis using WinNonlin v. 4.0.1 and the area under the curve (AUC₀₋₁₂₀) of NDMC was calculated. In vivo conversion to NDMC was calculated from the dose-normalized ratio of AUC₀₋₁₂₀ of NDMC following administration of pro-drug and NDMC, respectively.

The results are summarized in Table 1. The results indicated that the parent prodrugs were converted to NDMC in vivo. Furthermore, NDMC was detected in the brain.

TABLE 1
Conversion of prodrugs into NDMC in vivo.

| | | | Mean brain |
|---|---|---|---|
| | Plasma | % Compound | concentration |
| | AUC_{(0-120 min)} | Conversion | of NDMC at |
| Compound | of NDMC | In Vivo to | 120 min (SD) |
| Administered | (hr * nmol/L) | NDMC | (nmol/(kg) n = 3 |
| NDMC | 1298 | — | 255 (56) |
| Clozapine | 148 | 12 | <50 (—) |
| Example 28 | 24 | 3 | <10 (—) |
| Example 25 | 39 | 4 | 11 (0.4) |
| Example 29 | 51 | 4 | <10 (—) |
| Example 30 | 198 | 23 | 49 (12) |
| Example 3 | 465 | 44 | 234 (156) |

What is claimed is:

1. A compound of formula I: 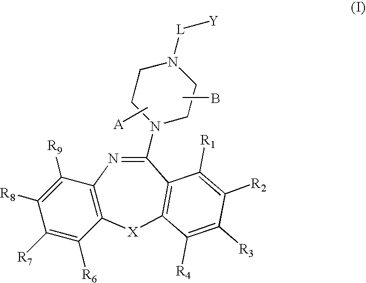
or a pharmaceutically acceptable salt thereof, wherein:
X is selected from the group consisting of NR¹⁰, O, CR¹⁰R¹¹, C═CR¹⁰R¹¹, and C═O;
A and B are independently selected from the group consisting of hydrogen and mono-substituted, poly-substituted or unsubstituted, cyclic or acyclic, straight or branched chain variants of the following residues: C₂-C₆ alkyl, C₂-C₆ alkenyl, C₂-C₆ alkynyl, and C₂-C₆ alkoxy;
A and B may optionally be bound together to form a cycloalkyl, heterocyclyl, heteroaryl, or aryl ring fused to the piperazine ring;
L is a cleavable linker moiety capable of being metabolically cleaved from the piperazine nitrogen;
Y is selected from the group consisting of hydrogen; halogen; cyano; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂, —NR¹⁰COR¹¹; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, acyl, arylalkyl, heteroarylalkyl, alkyloxycarbonyloxy, arylalkoxy, C₁-C₂₄ alkoxyalkyl, C₁-C₂₄ alkylthio, C₃-C₂₄ heterocycloalkyl-alkyl, and C₃-C₂₄ heterocycloalkenyl-alkyl; and mono-substituted, poly-substituted or unsubstituted variants of the following residues: acyloxy, aryloxycarbonyloxy, C₃-C₂₄ cycloalkyl, C₃-C₂₄ cycloalkenyl, C₂-C₂₄ cycloalkoxy, aryl, heteroaryl, C₂-C₂₄ heterocycloalkyl, C₂-C₂₄ heterocycloalkenyl, carbonyl, amino, aminocarbonyl, aminocarbonyloxy, nitro, azido, phenyl, hydroxyl, arylthio, carboxy, thio, acyl mono-radicals derived from naturally occurring amino acids or from lipoic acid, —CH₂CH₂SC(O)OR¹⁰, and —CH₂CH₂SC(O)R¹⁰, with the proviso that -L-Y is not unsubstituted alkyl;
R¹, R², R³, R⁴, R⁶, R⁷, R⁸, and R⁹ are each independently selected from the group consisting of hydrogen; halogen; —OH; —SH; —CN; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂; —NR¹⁰C(O)R¹¹; and mono-substituted, poly-substituted or unsubstituted, straight or branched chain variants of the following residues: C₁₋₆ alkyl, C₂₋₆ alkenyl, C₂₋₆ alkynyl, C₁₋₆ alkyloxy, C₁₋₆ heteroalkyl, C₁₋₆-alkoxyalkyl, C₁₋₆ alkylthio, C₁₋₆ perhaloalkyl, and C₁₋₆ perhaloalkoxy,
independently R¹ and R², R² and R³, R³ and R⁴, R⁶ and R⁷, R⁷ and R⁸, or R⁸ and R⁹ may optionally be taken together, along with the ring carbons to which they are attached, to form a five-membered or six-membered cycloalkyl, heterocyclyl, or heteroaryl ring, or a six-membered aryl ring,
independently R¹⁰ in X may optionally be bound to R⁴ or R⁶ to form a five-membered optionally substituted heterocyclyl or heteroaryl ring system;
R¹⁰, R¹¹, and R¹² are each independently selected from a group consisting of hydrogen and mono-substituted, poly-substituted or unsubstituted, straight or branched chain variants of the following residues: C₁₋₆ alkyl, C₃₋₆ cycloalkyl, C₂₋₆ alkenyl, C₂₋₆ alkynyl, arylalkyl, C₂₋₆alkylcarbonyl, C₂₋₆ alkoxycarbonyl, C₆₋₁₀ aryl, and C₅₋₁₀ heteroaryl.

2. The compound of claim 1, wherein L is selected such that the piperizine nitrogen together with L form a tertiary amine, a carbamate, a urea, an amide, an enamine, a sulfamide, a sulfonamide, an aminal, a hydrazine, or a hydroxylamine.

3. The compound of claim 1, wherein L is selected from the group consisting of carboxy, carbonyl, and alkoxy.

4. The compound of claim 3, wherein L is selected from the group consisting of —C(O)O—, —C(O)—, and —(CH₂)₂O—.

5. The compound of claim 1, wherein L is selected from a group consisting of —C(O)—, —C(O)O—, —C(O)NR¹⁰—, —NR¹⁰C(O)NR¹¹—, —NR¹⁰C(O)—, —NR¹⁰—, —CR¹⁰R¹¹OC(O)—, —CR¹⁰R¹¹C(O)O—, 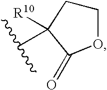
and

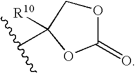

6. The compound of claim 1, wherein -L-Y is hydroxyalkyl.

7. The compound of claim 1, wherein Y is selected from a group consisting of hydrogen; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, phenyl-C₁₋₂-alkyl, C₃C₂₄ heterocycloalkyl-alkyl, and C₃-C₂₄ heterocycloalkenyl-alkyl; and mono-substituted, poly-substituted or unsubstituted variants of the following residues: C₃-C₂₄ cycloalkyl, C₃-C₂₄ cycloalkenyl, C₂-C₂₄ cycloalkoxy, C₂-C₂₄ heterocycloalkyl, C₂-C₂₄ heterocycloalkenyl, acyl mono-radicals derived from naturally occurring amino acids or from lipoic acid, —CH₂CH₂SC(O)OR¹⁰, and —CH₂CH₂SC(O)R¹⁰.

8. The compound of claim 1, wherein R² is H or F.

9. The compound of claim 1, wherein R⁸ is Cl, Br, or I.

10. The compound of claim 1, wherein R⁴ is Cl or Me.

11. The compound of claim 1, wherein X is NH.

12. The compound of claim 1, wherein X is O.

13. The compound of claim 1, selected from the group consisting of:

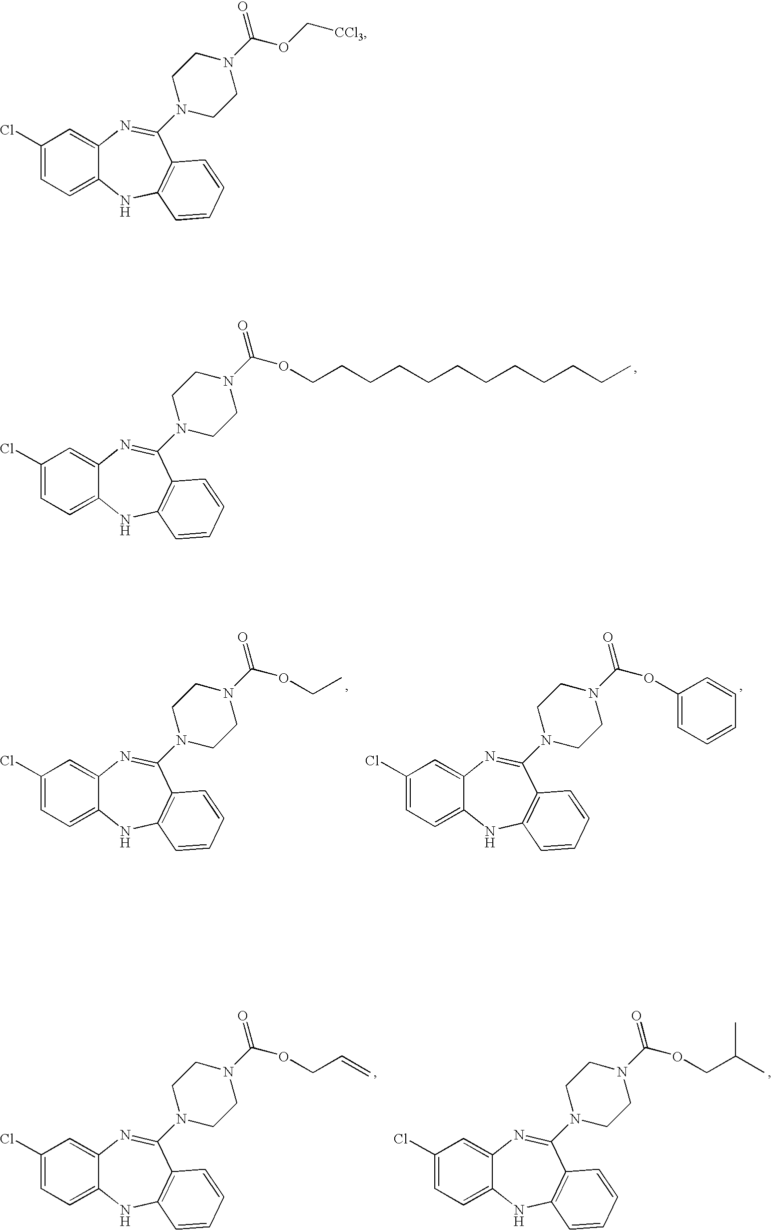

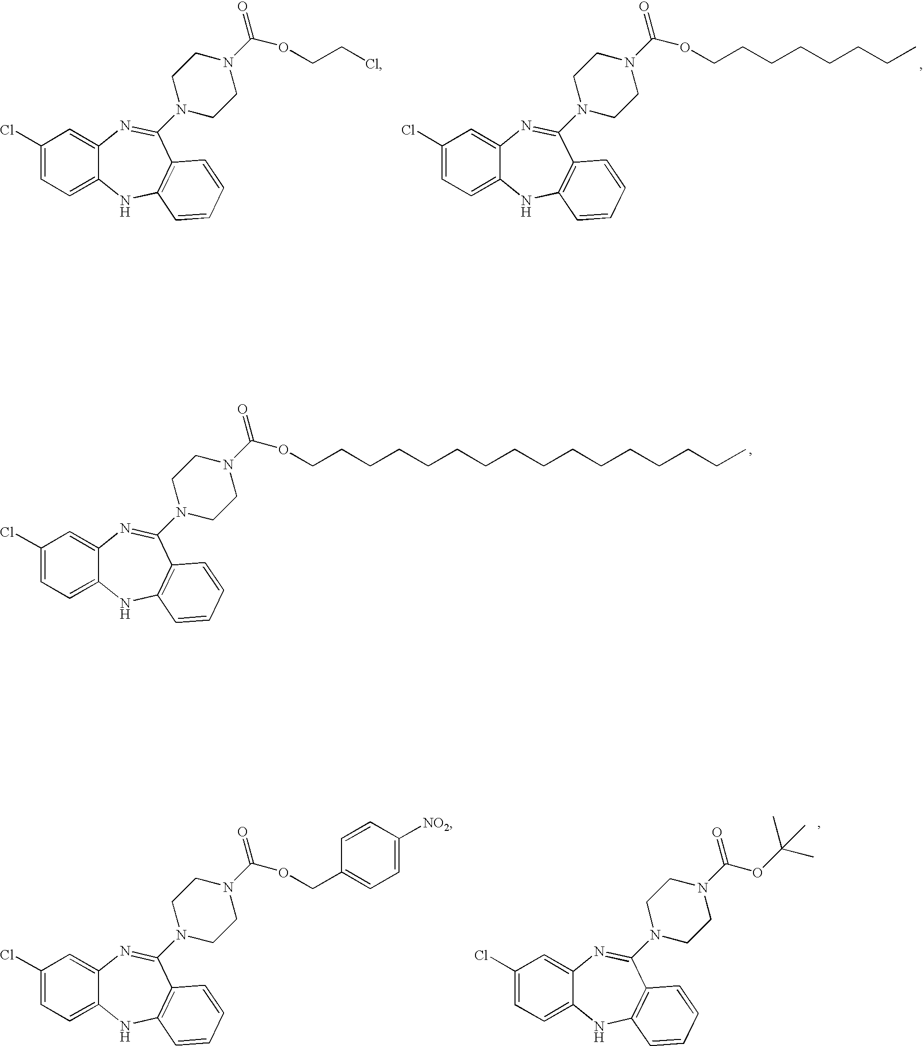

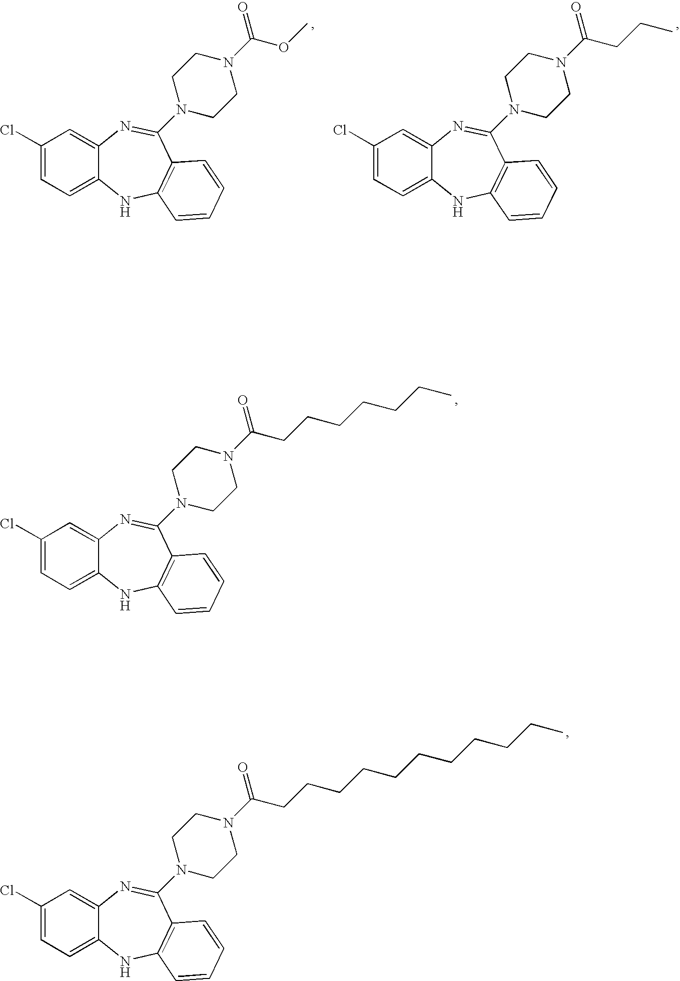

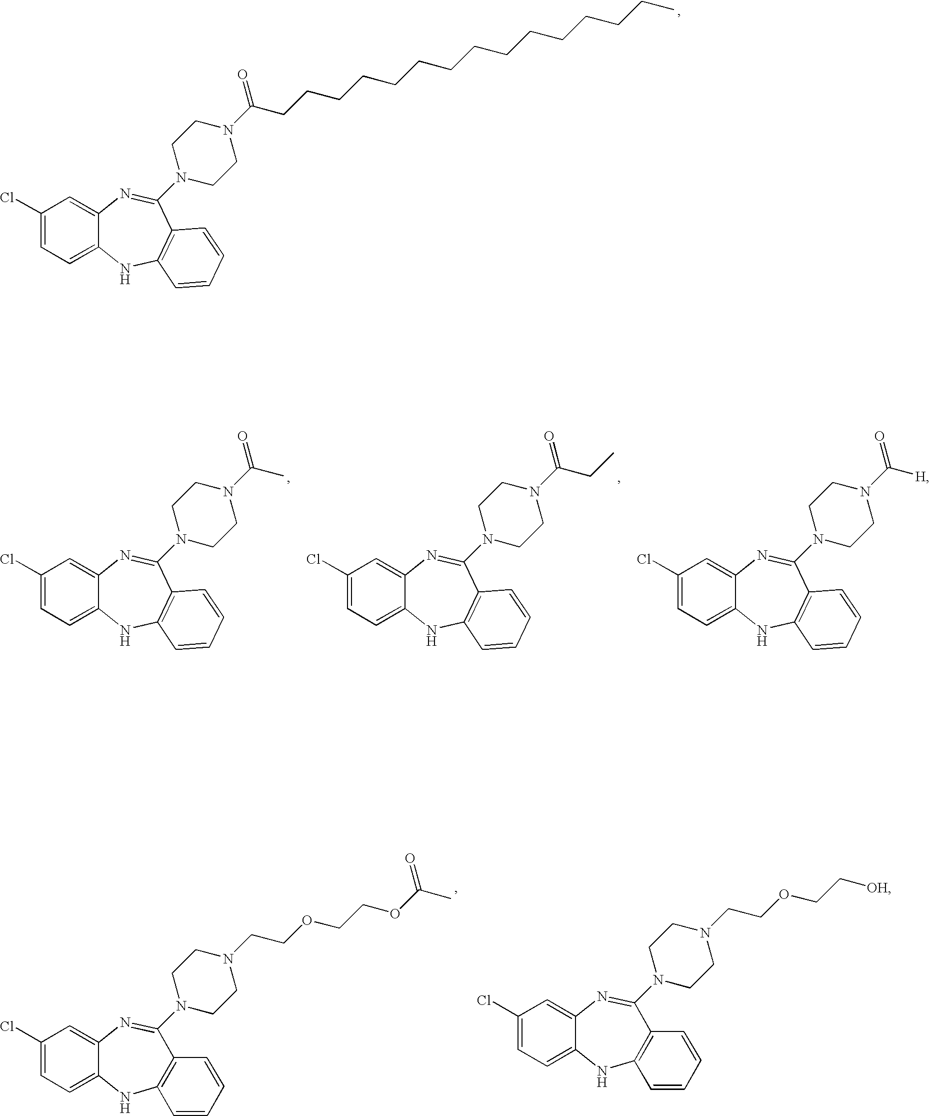

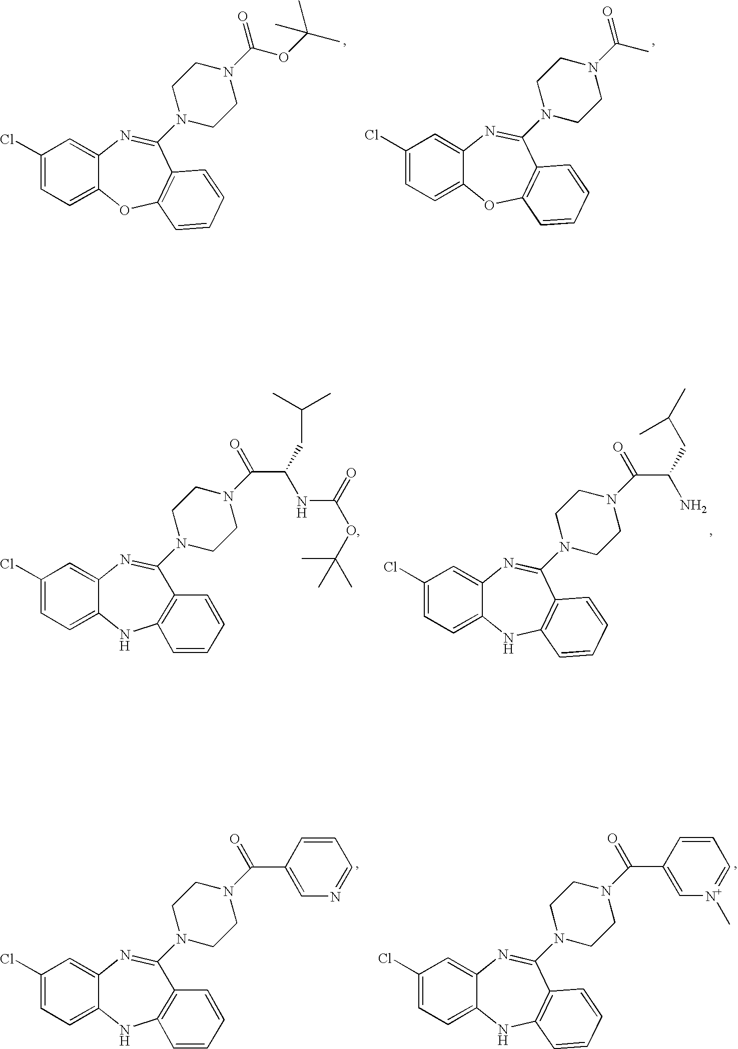

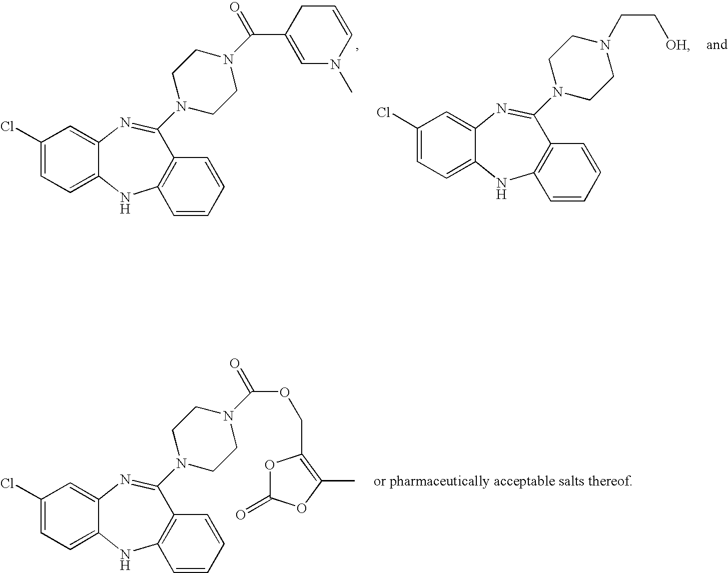

14. A compound of formula (II): 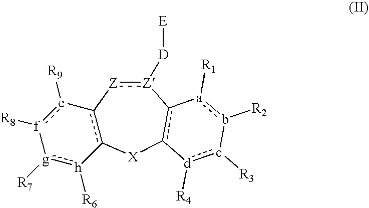
or a pharmaceutically acceptable salt thereof, wherein:
D is absent or is selected from the group consisting of —NH(CH₂)ₙ— and —(CH₂)ₙ—;
E is selected from the group consisting of: 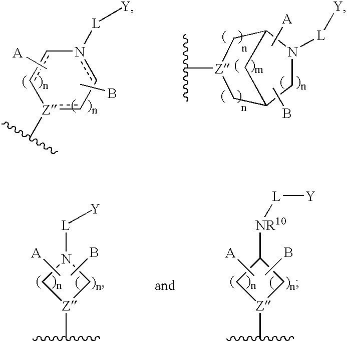
Z is nitrogen, CH, or CH₂;
Z′ is C or CH, wherein when Z′ is C, there is a double bond between Z and Z′ and wherein when Z′ is CH, there is a single bond between Z and Z′;
Z″ is N or CH;
each n is separately selected from the group consisting of 0, 1, 2, 3, and 4;
m is selected from the group consisting of 1, 2, and 3;
X is selected from the group consisting of NR¹⁰, O, CR¹⁰R¹¹, C═CR¹⁰R¹¹, and C═O;
A and B are independently selected from the group consisting of hydrogen and mono-substituted, poly-substituted or unsubstituted, cyclic or acyclic, straight or branched chain variants of the following residues: C₁-C₆ alkyl, C₂-C₆ alkenyl, C₂-C₆ alkynyl, and C₂-C₆ alkoxy;
A and B may optionally be bound together to form a fused cycloalkyl, heterocyclyl, heteroaryl, or aryl ring;
L is a cleavable linker moiety capable of being metabolically cleaved from the nitrogen to which it is attached;
Y is selected from the group consisting of hydrogen; halogen; cyano; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂, —NR¹⁰COR¹¹; mono-substituted, poly-substituted or unsubstituted, straight or branched chain variants of the following residues: C₁-C₂₄ alkyl, C₂-C₂₄ alkenyl, C₂-C₂₄ alkynyl, C₁-C₂₄ alkoxy, C₁-C₂₄ heteroalkyl, C₁-C₂₄ perhaloalkyl, C₁-C₂₄ perhaloalkoxy, acyl, arylalkyl, heteroarylalkyl, alkyloxycarbonyloxy, arylalkoxy, C₁-C₂₄ alkoxyalkyl, C₁-C₂₄ alkylthio, C₃-C₂₄ heterocycloalkyl-alkyl, and C₃-C₂₄ heterocycloalkenyl-alkyl; and mono-substituted, poly-substituted or unsubstituted variants of the following residues: acyloxy, aryloxycarbonyloxy, C₃-C₂₄ cycloalkyl, C₃-C₂₄ cycloalkenyl, C₂-C₂₄ cycloalkoxy, aryl, heteroaryl, C₂-C₂₄ heterocycloalkyl, C₂-C₂₄ heterocycloalkenyl, carbonyl, amino, aminocarbonyl, aminocarbonyloxy, nitro, azido, phenyl, hydroxyl, arylthio, carboxy, thio, acyl mono-radicals derived from naturally occurring amino acids or from lipoic acid, —CH₂CH₂SC(O)OR¹⁰, and —CH₂CH₂SC(O)R¹⁰, with the proviso that -L-Y is not unsubstituted alkyl;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R¹, R², R³, R⁴, R⁶, R⁷, R⁸, and R⁹ are each independently selected from the group consisting of hydrogen; halogen; —OH; —SH; —CN; —C(O)R¹⁰; —C(O)OR¹⁰; —C(O)NR¹⁰R¹¹; —NR¹²C(O)NR¹⁰R¹¹; —SO₂NR¹⁰R¹¹; —SO₂R¹⁰; —OSO₂R¹⁰; —NO₂; —NR¹⁰C(O)R¹¹; and mono-substituted, poly-substituted or unsubstituted, straight or branched chain variants of the following residues: C₁₋₆ alkyl, C₂₋₆ alkenyl, C₂₋₆ alkynyl, C₁₋₆ alkyloxy, C₁₋₆ heteroalkyl, C₁₋₆-alkoxyalkyl, C₁₋₆ alkylthio, C₁₋₆ perhaloalkyl, and C₁₋₆ perhaloalkoxy,
independently R¹ and R², R² and R³, R³ and R⁴, R⁶ and R⁷, R⁷ and R⁸, or R⁸ and R⁹ may optionally be taken together, along with the ring carbons to which they are attached, to form a five-membered or six-membered cycloalkyl, heterocyclyl, or heteroaryl ring, or a six-membered aryl ring,
independently R¹⁰ in X may optionally be bound to R⁴ or R⁶ to form a five-membered optionally substituted heterocyclyl or heteroaryl ring system;
R¹⁰, R¹¹, and R¹² are each independently selected from a group consisting of hydrogen and mono-substituted, poly-substituted or unsubstituted, straight or branched chain variants of the following residues: C₁₋₆ alkyl, C₃₋₆ cycloalkyl, C₂₋₆ alkenyl, C₂₋₆ alkynyl, arylalkyl, alkylcarbonyl, C₂₋₆ alkoxycarbonyl, C₆₋₁₀ aryl, and C₅₋₁₀ heteroaryl; and
any bond represented by a dashed and solid line represents a bond selected from the group consisting of a carbon-carbon single bond and a carbon-carbon double bond.

15. A method of modulating the activity of a muscarinic receptor, comprising administering to a subject a compound of claim 1 or 14.

16. The method of claim 15, wherein administration of the compound causes activation of the muscarinic receptor.

17. The method of claim 15, wherein administration of the compound causes inhibition of the muscarinic receptor.

18. The method of claim 15, wherein administration of the compound causes formation of a second compound in vivo that is an agonist of the muscarinic receptor.

19. The method of claim 15, wherein administration of the compound causes formation of a second compound in vivo that is an antagonist of the muscarinic receptor.

20. A method of treating or preventing a neuropsychiatric disorder, comprising administering to a subject a compound of claim 1 or 14.

21. The method of claim 20, wherein the neuropsychiatric disorder is selected from the group consisting of one or more of psychosis, cognitive impairment associated with psychosis, hallucination, delusion, disordered thought, behavioral disturbance, aggression, neuropathic pain, anhedonia, a psychiatric disturbance secondary to dementia or cognitive impairment, a behavioral disturbance secondary to dementia or cognitive impairment, schizophrenia, an idiopathic psychosis, anxiety, a sleep disorder, an appetite disorder, an affective disorder, Tourette's Syndrome, drug-induced psychosis, psychosis secondary to a neurodegenerative disorder, and cognitive impairment secondary to a neurodegenerative disorder.

22. The method of claim 21, wherein the affective disorder is selected from one or more of major depression, bipolar disorder, mania, flattening of affect, suicidality, and depression with psychotic features.

23. The method of claim 21, wherein the neurodegenerative disorder is selected from one or more of Alzheimer's and Huntington's disease.

24. A method of treating or preventing glaucoma, comprising administering to a subject a compound of claim 1 or 14.

25. A pharmaceutical composition, comprising a compound of claim 1 or 14.

26. The composition of claim 25, wherein the composition comprises one or more of a physiologically acceptable carrier, diluent, or excipient.
